(19) United States
(12) Patent Application Publication
(10) Pub. No.: US 2016/0068910 A1
(43) Pub. Date: Mar. 10, 2016

(54) Genetic Assay for Skin Pigmentation

(71) Applicant: Avon Products, Inc., Suffern, NY (US)

(72) Inventors: Yong Zhuang, Livingston, NJ (US); Uma Santhanam, Tenafly, NJ (US); John W. Lyga, Basking Ridge, NJ (US)

(21) Appl. No.: 14/843,279

(22) Filed: Sep. 2, 2015

Related U.S. Application Data
(60) Provisional application No. 62/048,683, filed on Sep. 10, 2014.

(51) Int. Cl. C12Q 1/68

(52) U.S. Cl. CPC C12Q 1/6883; C12Q 2600/158

(57) ABSTRACT

The present invention relates generally to the identification of genes involved in skin pigmentation and to screening methods for identifying cosmetic active agents that modulate skin pigmentation. The invention also relates to compositions for topical application to the skin that comprise modulators (i.e., downregulators and/or upregulators) of genes involved in skin pigmentation, and to methods for increasing or decreasing skin pigmentation by topically administering compositions of the invention to the skin.

### CROSS-REFERENCE TO RELATED APPLICATIONS

This application claims priority to U.S. Provisional Patent Application Ser. No. 62/048,683, filed Sep. 10, 2014, the entirety of which is incorporated herein for all purposes.

### FIELD OF INVENTION

The present invention relates generally to the identification of genes involved in skin pigmentation and to screening methods for identifying cosmetic active agents that modulate skin pigmentation. The invention also relates to compositions for topical application to the skin that comprise modulators (i.e., downregulators and/or upregulators) of genes involved in skin pigmentation, and to methods for increasing or decreasing skin pigmentation by topically administering compositions of the invention to the skin.

### BACKGROUND OF THE INVENTION

There is an increasing demand in the cosmetics industry to develop products that may be applied topically to the skin that improve or alter the condition and appearance of skin. Consumers are interested in mitigating, delaying, or reversing the dermatological signs of chronologically- or hormonally-aged skin, as well as skin aging due to environmental stress. During the aging process, the complexion of the skin, e.g., the color and appearance of the skin, deteriorates from aging and/or prolonged exposure to environmental stress, such as exposure to sunlight ultraviolet (UV) rays, and environmental insults.

In particular, aging can result in senile lentigos (age spots), which are hyperpigmented macules visible on the surface of the skin ranging in size from a few millimeters to more than a centimeter in diameter. Most frequently, age spots are present on UV-exposed areas of the skin, such as the face, back of the hands, and forearms, and usually manifest after the age of 40. Histological studies reveal that age spots have a hyperpigmented basal layer, an increased number of melanocytes and melanosomal protein, as well as altered keratinocyte differentiation and an increased inflammatory response.

Numerous cosmetic and medical treatments have been developed in an attempt to treat environmentally damaged, aging or aged skin. However, many of the cosmetics and treatments developed to treat hyperpigmentation (e.g., hydroquinone, kojic acid, etc.) are commonly associated with consumer discomfort, such as burning, itching, redness, stinging, crusting, swelling, and unusual discoloration. There is therefore a continuing need to further understand the pathways and molecular mechanisms that are associated with hyperpigmented skin. There is also a continuing need for effective topical (e.g., cosmetic and dermatological) treatments that address the visible signs of aging, including hyperpigmentation and age spots.

It is therefore an object of the invention to provide methods for screening to identify modulators of skin pigmentation that can be used in topical compositions to treat or prevent discoloration of the skin, such as hyperpigmentation and hypopigmentation. It is a further object of the invention to provide new compositions and methods for modulating (i.e., reducing or increasing) skin pigmentation.

The foregoing discussion is presented solely to provide a better understanding of nature of the problems confronting the art and should not be construed in any way as an admission as to prior art nor should the citation of any reference herein be construed as an admission that such reference constitutes “prior art” to the instant application.

### SUMMARY OF THE INVENTION

The present invention is based on the discovery that skin pigmentation is associated with the expression of a unique set of genes. In order to identify the set of genes that can be used to screen for these skin actives, the inventors compared gene expression in skin affected by age spots (lesional skin), skin adjacent to age spots (peri-lesional skin), and in skin that had been protected from the sun, exhibiting no age spots as explained in Example 1. Gene array analyses were conducted on mRNA isolated from the skin, and comparisons of gene expression levels were made between lesional skin and protected skin, and between lesional skin and peri-lesional skin. Gene expression was considered modulated between groups if there was a change in expression (whether upregulated or downregulated) of at least 1.3-fold from protected skin to lesional skin, and of at least 1.2 fold from peri-lesional skin to lesional skin. 482 genes were modulated in lesional skin relative to protected skin, and 118 genes were modulated in peri-lesional skin relative to lesional skin. A unique subset of 52 genes (see Table 1) was modulated in lesional skin relative to both peri-lesional and protected skin. This unique group of genes provides a gene signature for identifying cosmetic actives that can alter skin pigmentation. Specifically, the ability of a candidate substance to modulate skin pigmentation may be determined by contacting a skin cell (e.g., fibroblast, keratinocyte, an/or melanocyte) with the candidate substance and determining whether any of the 52 genes (e.g., two or more, three or more, four or more, etc.) are differentially expressed (i.e., upregulated or downregulated).

Accordingly, in one aspect of the invention, a method is provided for identifying active agents useful for improving the aesthetic appearance of skin, such as modulating skin pigmentation. The method generally comprises contacting a human skin cell (e.g., fibroblast, melanocyte, keratinocyte) with a candidate substance and measuring expression levels of at least two genes (e.g., at least 3 genes, at least 4 genes, at least 5 genes, at least 10 genes, etc.) selected from those listed in Table 1, relative to control (e.g., otherwise identical cells treated with a vehicle in the absence of the candidate substance). In other aspects, the candidate substance may be topically applied to skin (in vivo or ex vivo) to assess the modulation of those genes. The skin may be skin that has been protected from the sun, or from a hyperpigmented (e.g, age spot, freckle) or hypopigmented (e.g., pale patch) area of skin. It may then be determined if the expression of at least two of the measured genes is modulated (e.g., upregulated or downregulated) following contact with the candidate substance, relative to control. Expression of the genes in Table 1 may be assessed, although expression of any homolog, fragment or marker of the genes listed in Table 1 may be measured as well. Modulation (e.g., about a 1.5-fold, 2-fold, 3-fold, 4-fold, 5-fold, 10-fold, 12-fold, 15-fold, etc. change in expression relative to control or baseline) of two or more genes selected from Table 1 indicates that the candidate substance is a modulator of skin pigmentation (e.g., skin hyperpigmentation, hypopigmentation, discoloration, melasmic and chloasmic patches, freckles, after-burn scars, suntan, etc.). The method may also comprise extracting nucleic acids (e.g., mRNA, DNA, etc.) from skin cells that have not been and skin cells that have been exposed to a candidate substance. Typically, complementary nucleic acids (e.g., cDNA, cRNA, etc.) are synthesized from the extracted nucleic acids in enzyme catalyzed reactions known to those skilled in the art. Gene expression levels may then be quantified using methods routine to those skilled in the art (e.g., quantitative PCR, Northern blot assays, RNA protection assays, etc.). In some aspects, the method involves measuring gene expression levels with a gene array. When a gene array is used to assess gene expression, nucleic acids complementary to the genes may be contacted with a solid phase gene panel (e.g., comprising glass, silicon, nylon, etc.) that comprises immobilized oligonucleotides (e.g., primers or probes) that hybridize specifically to nucleic acids corresponding to two or more genes selected from Table 1 (e.g., that hybridize to cDNA or cRNA complementary to a portion of those genes). The hybridization may then be detected and quantified by methods known in the art (e.g., using fluorophore-labelled cDNA or cRNA) to quantify gene expression levels.

In another aspect of the invention, a gene array is provided for active agents that can modulate skin pigmentation. The gene array typically comprises a solid phase having immobilized thereon a plurality of oligonucleotides (e.g., primers, probes, etc.) that hybridize specifically to nucleic acid sequences corresponding to or complementary to genes listed in Table 1. In some aspects, a gene array (e.g., gene chip, biochip, gene panel) is provided wherein at least 50%, (e.g., at least 60%, at least 70%, at least 80%, at least 85%, at least 90%, at least 95%, or at least 99%) of the nucleic acids on the gene array comprise oligonucleotides (e.g., primers, probes that may be less than about 60, less than about 50, less than about 40, less than about 30, less than about 20, or less than about 10 nucleotides in length) that hybridize specifically to nucleic acids corresponding to or complementary to genes listed in Table 1. In some implementations, the gene array will comprise nucleic acids that correspond to or are complementary to at least two, or at least three, or at least four, or at least five, or at least six, or at least seven, or at least eight, or at least nine, or at least ten genes from Table 1. In other implementations, the gene array will comprise at least fifteen, or at least twenty, or at least twenty-five, or at least thirty, or at least thirty-five, or at least forty, or at least forty-five genes from Table 1.

In another aspect of the invention, methods are provided for reducing skin pigmentation (e.g., age spots, freckles, suntan, etc.), as well as for increasing skin pigmentation (e.g., in pale patches of skin associated with vitiligo or albinism). The methods generally comprise topically applying to an area of skin an effective amount of a downregulator (e.g., monocolonal antibody, siRNA, antisense, etc.) and/or an upregulator (e.g., a nuclear receptor agonist) of two or more (e.g., three or more, four or more, five or more, etc.) of the genes identified in Table 1.

In another aspect of the invention, cosmetic compositions (e.g., in a cosmetically acceptable vehicle) are provided that comprise one or more downregulators and/or upregulators identified by the screening methods of the invention.

These and other aspects of the present invention will be better understood by reference to the following detailed description and appended claims.

### DETAILED DESCRIPTION

All terms used herein are intended to have their ordinary meaning unless otherwise provided. All ingredient amounts provided herein are by weight percent of the total composition unless otherwise indicated. As used herein, the term “consisting essentially of” is intended to limit the invention to the specified materials or steps and those that do not materially affect the basic and novel characteristics of the claimed invention, as understood from a reading of this specification.

As used herein, the term “expression levels” refers to an amount of a gene and/or protein that is expressed in a cell (e.g., number of mRNA copies made). As used herein, a “gene” includes a polynucleotide containing at least one open reading frame that is capable of encoding a particular polypeptide. As used herein, the term “polynucleotide” includes “oligonucleotide” and includes polymeric forms of nucleotides (nucleic acids) of any length, either deoxyribonucleotides or ribonucleotides, or analogs thereof, including, without limitation, mRNA, DNA, cDNA, cRNA, primers, probes, and the like.

The present invention is based on the discovery of a novel set of genes associated with aberrant skin pigmentation. This novel set of genes is useful for identifying agents that modulate pigmentation in the skin.

In one embodiment, a method is provided for identifying active agents useful for improving the aesthetic appearance of skin, including, without limitation, modulating skin pigmentation. The method generally comprises contacting a human skin cell with a candidate substance, and measuring the expression levels of two or more genes selected from the group consisting of the genes listed in Table 1, relative to expression levels of the same genes in a control sample (baseline), by which is meant otherwise identical cells treated with a vehicle in the absence of the candidate substance. In other embodiments, the candidate substance may be topically applied to skin (in vivo or ex vivo) to assess the modulation of those genes. Modulation of two or more genes (or any homolog, fragment or marker of the genes) in Table 1 indicates that the candidate substance is an active agent that modulates skin pigmentation.

Any suitable human skin cells may be used such as, for example, fibroblasts, melanocytes, and/or keratinocytes.

In embodiments where the candidate is topically applied to skin, any portion of human skin tissue may be contacted with a candidate substance to assess gene expression levels in that tissue, such as skin from the face, neck, arms, legs, buttocks, chest, etc. The skin cells may be obtained from skin that has been protected from the sun, or from a hyperpigmented (e.g, age spot, freckle) or a hypopigmented (e.g., pale patch) area of skin. Gene expression may be measured from any skin cell type, such as, for example, fibroblasts melanocytes, or keratinocytes.

The candidate substance may be applied to the skin or skin cells topically, for example in the form of a cream, lotion, ointment, patch, etc. The candidate substance will be in contact with the skin or with the skin cells for a sufficient length of time to provide a measurable change in gene expression levels, which will typically be at least half an hour, at least one hour, and more typically from about 12 hours to about 72 hours. However, in some embodiments, the candidate substance may be in contact with the skin or skin cells for about one or more weeks, or for about one or more months. In some embodiments, other forms of skin treatment may be assessed by the screening methods of the invention, such as laser treatments, phototherapy, thermolysis treatments, electromagnetic radiation, or other treatments involving use of current, such as radio frequency current.

Following treatment of skin with a candidate substance, a biological sample, such as a sample of the treated skin, may be obtained. Biological samples may also be obtained from skin not treated with a candidate substance. A biological sample may include any sample of biological tissue or fluid that comprises nucleic acids. Skin cells may be obtained from at least one portion of the treated (or untreated) skin, for example, by biopsy (e.g., punch biopsy), by fine-needle aspiration, by cell scraping, etc., so that nucleic acids may be extracted from the biological sample.

Nucleic acids (e.g., mRNA, DNA) may be extracted from skin cells or biological samples by any conventional methods, so that gene expression levels may be measured. Typically, prior to gene expression quantification procedures, complementary nucleic acids (e.g., cDNA or cRNA) are synthesized from the extracted nucleic acids in enzymes catalyzed reactions known to those skilled in the art.

Gene expression levels may be measured by any suitable technique for detection and quantitation of polynucleotides (e.g., mRNA, DNA, microRNA), such as, for example, quantitative polymerase chain reaction (QPCR), real-time QPCR, reverse transcription PCR (RT-PCR), as are well-known in the art. As described in detail in U.S. Pat. Nos. 7,101,663 and 7,662,561, the disclosures of which are hereby incorporated by reference, a quantitative reverse transcriptase polymerase chain reaction (QRT-PCR) for detecting mRNA may include the steps of: (a) incubating an RNA sample from the cellular lysate with a reverse transcriptase and a high concentration of a target sequence-specific reverse transcriptase primer under conditions suitable to generate cDNA; (b) subsequently adding suitable polymerase chain reaction (PCR) reagents to the reverse transcriptase reaction, including a high concentration of a PCR primer set specific to the cDNA and a thermostable DNA polymerase to the reverse transcriptase reaction; and (c) cycling the PCR reaction for a desired number of cycles and under suitable conditions to generate PCR products (“amplicons”) specific to the cDNA. The products of the QRT-PCR process may be compared after a fixed number of PCR cycles to determine the relative quantity of the RNA species as compared to a given reference gene, for example, GAPDH (glyceraldehyde-3-phosphate dehydrogenase). More typically, the progress of the PCR reaction is monitored by analyzing the relative rates of amplicon production for each PCR primer set, for example, by (1) non-specific fluorescent dyes that intercalate with any double-stranded DNA, and/or (2) sequence-specific DNA probes consisting of oligonucleotides that are labeled with a fluorescent reporter which permits detection only after hybridization of the probe with its complementary DNA target.

In some embodiments, gene expression levels may be assessed with a gene array (i.e., gene microarray, gene panel, gene chip). When a gene array is used to assess gene expression levels, complementary nucleic acids (e.g., cDNA, cRNA) that are synthesized from extracted nucleic acids, are contacted with a solid phase having immobilized thereon a plurality of oligonucleotides (e.g., primers, probes) that hybridize specifically to nucleic acids corresponding to or complementary to genes listed in Table 1. Generally, the oligonucleotides are affixed via a linking chemistry to a solid substrate, such as glass or silicon. Primers and probes for detecting the genes identified in Table 1 may be designed by any routine methods in the art, and may be polynucleotides of any suitable length for detecting any of the two or more genes from Table 1, and may be less than 10, less than 15, less than 20, less than 30, less than 40, less than 50, less than 75, less than 100, less than 200, less than 500, or more than 500 nucleotides in length. The hybridization may then be detected and quantified by methods known in the art (e.g., using fluorophore-labelled cDNA or cRNA) to quantify gene expression levels.

Other suitable non-limiting methods for measuring gene expression include RNA in situ hybridization, RNAse protection assays, Northern blot assays, serial analysis of gene expression (SAGE analysis), immunohistochemistry assays, competitive-binding assays, gene expression analysis by massively parallel signature sequencing (MPSS), etc.

In some embodiments, expression of proteins or polypeptides produced by two or more genes identified in Table 1 may be useful for identifying modulators of the aesthetic appearance of skin, such as skin pigmentation. Any routine methods for assessing protein or polypeptide expression may be used, such as those that utilize antibodies, including flow cytometry, immunohistochemistry, ELISA, Western blot, Northwestern blot, and immunoaffinity chromatograpy. Antibodies may be monoclonal, polyclonal, or any antibody fragment including an Fab, F(ab)₂, Fv, scFv, phage display antibody, peptibody, multispecific ligand, or any other reagent with specific binding to a target. Other suitable methods for assessing protein expression include HPLC, mass spectrometry, protein microarray analysis, PAGE analysis, isoelectric focusing, 2-D gel electrophoresis, and enzymatic assays.

Gene expression levels (or protein expression levels) obtained from skin cells treated with a candidate substance may be compared to gene expression levels from otherwise identical cells treated with a vehicle, in the absence of the candidate substance (control) to determine the relative degree of modulation of the genes, which may comprise upregulation or downregulation of those genes. In some embodiments, the candidate substance will result in upregulation or downregulation of gene expression that is about a 1.3-fold change, about a 1.5-fold change, about a 2-fold change, about a 3-fold change, about a 4-fold change, about a 5-fold change, about a 6-fold change, about a 7-fold change, about an 8-fold change, about a 9-fold change, about a 10-fold change, about a 15-fold change, or about a 20-fold change, relative to control gene expression. Candidate substances meeting these criteria may be selected for use of for further evaluation.

In some embodiments, the expression levels of at least 3 genes, at least 4 genes, at least 5 genes, at least 6 genes, at least 7 genes, at least 8 genes, at least 9 genes, at least 10 genes, at least 15 genes, or at least 20 genes selected from Table 1 is modulated in a skin cell, following contact with a candidate substance, relative to control. In some embodiments, the foregoing genes will be genes other than those selected from the group consisting of: tyrosinase, tyrosinase-related protein-1, premelanosome protein, melan-A endothelin receptor type B, keratin 6B, genes relating to melanogenesis, genes relating to inflammation, and genes relating to fatty acid metabolism.

In some embodiments, the expression levels of at least one gene, or at least 2 genes, or at least 3 genes, or at least 4 genes, or at least 5 genes listed in Table 1 is upregulated in a skin cell, following contact with a candidate substance, relative to control. In another embodiment, the expression levels of at least one gene, or at least 2 genes, or at least 3 genes, or at least 4 genes, or at least 5 genes listed in Table 1 is downregulated in a skin cell, following contact with a candidate substance, relative to control. In another embodiment, the expression levels of at least one gene listed in Table 1 is upregulated and the expression levels of at least one gene listed in Table 1 is downregulated in a skin cell, following contact with a candidate substance, relative to control. In some embodiments, the foregoing genes will be genes other than those selected from the group consisting of: tyrosinase, tyrosinase-related protein-1, premelanosome protein, melan-A endothelin receptor type B, keratin 6B, genes relating to melanogenesis, genes relating to inflammation, and genes relating to fatty acid metabolism.

In one embodiment, expression levels of at least one of the genes modulated in a skin cell following contact with a candidate substance, relative to control is selected from the group consisting of: homeobox D10; transient receptor potential cation channel, subfamily M, member 1; cytochrome P450, family 39, subfamily A, polypeptide 1; and mucolipin 3; homeobox D11; hemicentin 1; G protein-coupled receptor 143; and long intergenic non-protein coding RNA 518.

In some embodiments, expression levels of at least one gene involved in melanogenesis, inflammation, fatty acid metabolism, cellular movement, cell growth and proliferation, cellular development, cell death and survival, amino acid metabolism, and/or cellular function and maintenance, is modulated following contact with a candidate substance, relative to control.

In one embodiment, gene expression levels of tyrosinase are not measured. In another embodiment, gene expression levels of tyrosinase-related protein-1 are not measured. In another embodiment, gene expression levels of premelanosome protein are not measured. In one embodiment, gene expression levels of melan-A are not measured. In another embodiment, gene expression levels of endothelin receptor type B are not measured. In another embodiment, gene expression levels of keratin 6B are not measured. In another embodiment, gene expression levels of genes relating to melanogenesis, inflammation, or fatty acid metabolism are not measured.

The invention also provides a gene array (i.e., gene microarray, gene panel, gene chip, biochip) for screening for active agents that can modulate skin pigmentation. The gene array may comprise a solid phase having immobilized thereon a plurality of oligonucleotides (e.g., primers, probes, cDNA) that hybridize specifically to nucleic acids corresponding to or complementary to two or more genes from Table 1. Generally, the oligonucleotides are affixed or attached via a linking chemistry to a solid substrate (e.g., glass, silicon, bead, nylon membrane, etc.). The oligonucleotides may be affixed to the solid substrate so that the attachment is stable under conditions of binding, washing, analysis, and removal. In one embodiment, the gene array comprises oligonucleotides that hybridize with nucleic acids corresponding to or complementary to at least 2, at least 3, at least 4, at least 5, at least 6, at least 7, at least 8, at least 9, at least 10, at least 15, at least 20, at least 30, at least 40, or at least 50 of the genes listed in Table 1. In one embodiment, the gene array comprises oligonucleotides that hybridize with nucleic acids corresponding to or complementary to all of the genes listed in Table 1. In some embodiments, the foregoing genes will be genes other than those selected from the group consisting of: tyrosinase, tyrosinase-related protein-1, premelanosome protein, melan-A endothelin receptor type B, keratin 6B, genes relating to melanogenesis, genes relating to inflammation, and genes relating to fatty acid metabolism.

In some embodiments, a gene array is provided wherein at least 50%, at least 60%, at least 70%, at least 80%, at least 85%, at least 90%, at least 95%, or at least 99% of the nucleic acids on the gene panel comprise oligonucleotides (e.g., primers, probes) that hybridize specifically to nucleic acids corresponding to or complementary to at least one of the genes listed in Table 1.

Oligonucleotides (e.g., primers, probes) affixed to the solid substrate of the gene array for detecting the genes identified in Table 1 may be designed by any routine methods in the art, and may be polynucleotides of any suitable length for detecting any of the two or more genes from Table 1. Such primers or probes may be single-stranded or double-stranded, and may be less than 10, less than 15, less than 20, less than 30, less than 40, less than 50, less than 75, less than 100, less than 200, less than 500, or more than 500 nucleotides in length. The hybridization may then be detected and quantified by methods known in the art (e.g., using fluorophore-labelled cDNA or cRNA) to quantify gene expression levels.

Cosmetic compositions are also provided, comprising an effective amount of one or more active ingredients that downregulate and/or upregulate one or more of the genes (or any homolog, fragment or marker of the genes) identified in Table 1. Cosmetic compositions may be formulated with other cosmetically acceptable components, such as a vehicle, for topical application to the skin. In some embodiments, the foregoing genes will be genes other than those selected from the group consisting of: tyrosinase, tyrosinase-related protein-1, premelanosome protein, melan-A endothelin receptor type B, keratin 6B, genes relating to melanogenesis, genes relating to inflammation, and genes relating to fatty acid metabolism.

Suitable upregulators or downregulators may be any substance, including, without limitation, organic molecules; biomolecules (e.g., peptides, proteins, antibodies, nucleic acid oligomers, etc.); and combinations of substances, such as botanical extracts.

Downregulators may be, without limitation, inhibitors or antagonists, which are, for example, compounds that bind to, partially or totally block stimulation, decrease, prevent, delay activation, inactivate, desensitize, or downregulate expression levels of one or more of the genes identified in Table 1. In some embodiments, genes may be downregulated with an antibody, such as a monoclonal antibody, or an antibody/monoclonal antibody fragment. In other embodiments, genes may be downregulated through gene silencing, such as by employing antisense or short-interfering RNA (siRNA).

siRNA is a gene silencing process that uses double-stranded RNA that is generally less than about 50 base pairs long, and has a sequence complementary to the mRNA that is targeted. In some embodiments, the siRNA is between about 10 and about 50 base pairs, between about 20 and about 40 base pairs, or between about 25 and about 35 base pairs. siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly applied to the skin and enter cells, either by passive uptake or a delivery system of choice.

Upregulators may be, without limitation, activators or agonists, which are compounds that, for example, bind to, stimulate, increase, open, activate, facilitate, enhance activation, sensitize, or upregulate expression levels of one or more of the genes identified in Table 1.

In one embodiment, cosmetic compositions are provided comprising one or more downregulators and/or one or more upregulators of one or more of the genes selected from the group consisting of: MX dynamin-like GTPase 2; chromosome 10 open reading frame 90; myosin VA (heavy chain 12, myoxin); plexin C1; mucolipin 3; oculocutaneous albinism II; melan-A; 2′-5′-oligoadenylate synthetase 2, 69/71 kDa; G protein-coupled receptor 143; phosphatidic acid phosphatase type 2 domain containing 1A; epithelial membrane protein 3; paired box 3; solute carrier family 1 (glutamate/neutral amino acid transporter), member 4; long intergenic non-protein coding RNA 518; ectonucleotide pyrophosphatase/phosphodiesterase 2; neuronal regeneration related protein; CD24 molecule; protocadherin 7; serpin peptidase inhibitor, clade B (ovalbumin), member 12; transient receptor potential cation channel, subfamily M, member 1; endothelin receptor type B; WD repeat domain 63; tolloid-like 1; tyrosinase-related protein 1; premelanosome protein; hemicentin 1; homeobox D11; keratin 6B; insulin-like growth factor binding protein 3; Fraser extracellular matrix complex subunit 1; FRAS1 related extracellular matrix protein 2; small proline-rich protein 1A; follicle stimulating hormone receptor; ATPase, H+/K+ transporting, nongastric, alpha polypeptide; and homeobox D10. Downregulators of these genes generally will result in a decrease in skin pigmentation, and upregulators of these genes generally will result in an increase in skin pigmentation.

In another embodiment, cosmetic compositions are provided comprising one or more downregulators and/or one or more upregulators of one or more of the genes selected from the group consisting of: cytochrome P450, family 39, subfamily A, polypeptide 1; actin, alpha, cardiac muscle 1; periostin, osteoblast specific factor; SLIT and NTRK-like family, member 6; ribosomal protein S6 kinase, 90 kDa, polypeptide 6; coagulation factor II (thrombin) receptor; actin, alpha, cardiac muscle 1; fibronectin leucine rich transmembrane protein 3; ring finger protein 180; nicotinamide nucleotide adenylyltransferase 3; olfactory receptor, family 2, subfamily T, member 2; zinc finger protein 204, pseudogene; solute carrier family 38, member 4; solute carrier family 25, member 27; kinesin family member 21A; serine peptidase inhibitor, and Kazal type 1. Downregulators of these genes generally will result in an increase in skin pigmentation, and upregulators of these genes generally will result in a decrease in skin pigmentation.

In some embodiments, the screening methods of the invention comprise measuring expression levels of one or more of the following genes following contact of a skin cell to a candidate substance, relative to a control: olfactory receptor, family 2, subfamily T, member 35; myxovirus (influenza virus) resistance 2 (mouse); and Fraser syndrome 1. An upregulator and/or downregulator of any one or more of these genes may also be present in a composition of the invention, and methods for modulating skin pigmentation or improving the aesthetic appearance of skin may comprise administering a composition comprising an upregulator and/or a downregulator of one or more of these genes.

The compositions according to the invention can be formulated in a variety of forms for topical application and may comprise from about 0.00001% by weight to about 90% by weight of one or more of the active ingredients (e.g., upregulators or downregulators of one or more genes identified in Table 1) identified by the screening methods of the invention. Typically the cosmetic compositions will comprise from about 0.0001% by weight to about 25% by weight, and more preferably from about 0.001% by weight to about 1% by weight of the active ingredient. In one embodiment, the active will comprise from about 0.01% by weight to about 0.1% by weight or to 0.5% by weight of the cosmetic composition. In another embodiment, the active will comprise from about 0.001% by weight to about 5% by weight of the composition. The compositions will comprise an effective amount of one or more of the active ingredients which is meant an amount sufficient to treat, prevent, ameliorate, forestall, and/or reduce one or more signs of skin aging or otherwise improve the aesthetic appearance of human skin, in a given area of skin when topically applied thereto. In particular, the compositions may comprise an amount of one or more of the active ingredients effective to treat hyperpigmentation or hypopigmentation.

The cosmetic composition may be formulated in a variety of product forms, such as, for example, a lotion, cream, serum, spray, aerosol, cake, ointment, essence, gel, paste, patch, pencil, towelette, mask, stick, foam, elixir, concentrate, and the like, particularly for topical administration.

Cosmetically acceptable vehicles may include water; vegetable oils; mineral oils; esters such as octal palmitate, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and biphenyl alcohol; isoparaffins such as isooctane, isododecane and is hexadecane; silicone oils such as cyclomethicone, dimethicone, dimethicone cross-polymer, polysiloxanes and their derivatives, preferably organomodified derivatives; hydrocarbon oils such as mineral oil, petrolatum, isoeicosane and polyisobutene; polyols such as propylene glycol, glycerin, butylene glycol, pentylene glycol and hexylene glycol; waxes such as beeswax and botanical waxes; or any combinations or mixtures of the foregoing. Aqueous vehicles may include one or more solvents miscible with water, including lower alcohols, such as ethanol, isopropanol, and the like.

The cosmetically acceptable vehicle may be in the form of an emulsion. Non-limiting examples of suitable emulsions include water-in-oil emulsions, oil-in-water emulsions, silicone-in-water emulsions, water-in-silicone emulsions, wax-in-water emulsions, water-oil-water triple emulsions or the like having the appearance of a cream, gel or microemulsions. The emulsion may include an emulsifier, such as a nonionic, anionic or amphoteric surfactant, typically in an amount from about 0.001% to about 5% by weight.

The compositions of the invention are typically suitable for topical application to the human integumentary system, including without limitations skin, nails, hair, etc. The site of application to skin may be skin of the face, lips, hands, chest, etc.

In some embodiments, the compositions may include additional skin actives such as, but are not limited to, botanicals, other keratolytic agents, desquamating agents, keratinocyte proliferation enhancers, collagenase inhibitors, elastase inhibitors, depigmenting agents, anti-inflammatory agents, steroids, anti-acne agents, antioxidants, thiodipropionic acid or esters thereof, and advanced glycation end-product (AGE) inhibitors. Exemplary anti-aging components include, without limitation, botanicals (e.g., Butea Frondosa extract); thiodipropionic acid (TDPA) and esters thereof; retinoids (e.g., all-trans retinoic acid, 9-cis retinoic acid, phytanic acid and others); hydroxy acids (including alpha-hydroxyacids and beta-hydroxyacids), salicylic acid and salicylates; other exfoliating agents (e.g., glycolic acid, 3,6,9-trioxaundecanedioic acid, etc.), estrogen synthetase stimulating compounds (e.g., caffeine and derivatives); compounds capable of inhibiting 5 alpha-reductase activity (e.g., linolenic acid, linoleic acid, finasteride, and mixtures thereof); barrier function enhancing agents (e.g., ceramides, glycerides, cholesterol and its esters, alpha-hydroxy and omega-hydroxy fatty acids and esters thereof, etc.); collagenase inhibitors; and elastase inhibitors; to name a few. In one embodiment, the composition comprises N-Acetyl Tyrosinamide.

Exemplary retinoids include, without limitation, retinoic acid (e.g., all-trans or 13-cis), and derivatives thereof, retinaldehyde, retinol (Vitamin A) and esters thereof, such as retinol palmitate, retinol acetate and retinol propionate, and salts thereof. Particular mention may be made of retinol. Retinoids will typically be included in amounts from about 0.0001% to about 5% by weight, more typically from about 0.01% to about 2.5% by weight or from about 0.1% to about 1.0% by weight. Compositions according to this embodiment will typically include an antioxidant such as ascorbic acid and/or BHT and/or a chelating agent such as EDTA or a salt thereof.

In another embodiment, the topical compositions of the present invention may also include one or more of the following: a skin penetration enhancer, an emollient, a humectant, a skin plumper, an optical diffuser, a sunscreen, an additional exfoliating agent, an antioxidant, and a pH adjuster.

An emollient provides the functional benefits of enhancing skin smoothness and reducing the appearance of fine lines and coarse wrinkles. Examples include isopropyl myristate, petrolatum, isopropyl lanolate, silicones (e.g., methicone, dimethicone), oils, mineral oils, fatty acid esters, or any mixtures thereof. The emollient may be preferably present from about 0.1 wt % to about 50 wt % of the total weight of the composition.

A skin plumper serves as a collagen enhancer to the skin. An example of a suitable, and preferred, skin plumper is palmitoyl oligopeptide. Other skin plumpers are collagen and/or other glycosaminoglycan (GAG) enhancing agents. When present, the skin plumper may comprise from about 0.1 wt % to about 20 wt % of the total weight of the composition.

A sunscreen for protecting the skin from damaging ultraviolet rays may also be included. Preferred sunscreens are those with a broad range of UVB and UVA protection, such as octocrylene, avobenzone (Parsol 1789), octyl methoxycinnamate, octyl salicylate, oxybenzone, homosylate, benzophenone, camphor derivatives, zinc oxide, and titanium dioxide. When present, the sunscreen may comprise from about 0.01 wt % to about 70 wt % of the composition.

Suitable exfoliating agents include, for example, alpha-hydroxyacids, beta-hydroxyacids, oxaacids, oxadiacids, and their derivatives such as esters, anhydrides and salts thereof. Suitable hydroxy acids include, for example, glycolic acid, lactic acid, malic acid, tartaric acid, citric acid, 2-hydroxyalkanoic acid, mandelic acid, salicylic acid and other derivatives thereof. A preferred exfoliating agent is glycolic acid. When present, the exfoliating agent may comprise from about 0.1 wt % to about 80 wt % of the composition.

An antioxidant functions, among other things, to scavenge free radicals from skin to protect the skin from environmental aggressors. Examples of antioxidants that may be used in the present compositions include compounds having phenolic hydroxy functions, such as ascorbic acid and its derivatives/esters; beta-carotene; catechins; curcumin; ferulic acid derivatives (e.g., ethyl ferulate, sodium ferulate); gallic acid derivatives (e.g., propyl gallate); lycopene; reductic acid; rosmarinic acid; tannic acid; tetrahydrocurcumin; tocopherol and its derivatives; uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. In one particular embodiment, the inventive compositions will include TDPA or an ester thereof (e.g., dilauryl thiodipropionic acid), and/or an alpha hydroxyl acid (glycolic acid) and/or beta hydroxyl acid (salicylic acid or a derivative). Compositions of the present invention may comprise an antioxidant, which may comprise from about 0.001 wt % to about 10 wt %, or from about 0.01 wt % to about 5 wt %, of the total weight of the composition.

Other conventional additives include: vitamins, such as tocopherol and ascorbic acid; vitamin derivatives such as ascorbyl monopalmitate; thickeners such as hydroxyalkyl cellulose; gelling agents; structuring agents; metal chelating agents such as EDTA or salts thereof; pigments; colorants; and pH adjusters. The composition may optionally comprise other components known to those skilled in the art including, but not limited to, film formers, moisturizers, minerals, viscosity and/or rheology modifiers, anti-acne agents, insect repellents, skin cooling compounds, skin protectants, lubricants, fragrances, preservatives, stabilizers, and mixtures thereof. In addition to the foregoing, the cosmetic compositions of the invention may contain any other compound for the treatment of skin disorders. The conventional additives, actives, adjuvants, and excipients set forth in the preceding paragraphs are present in the compositions in amounts suitable to obtain their intended purpose and effect, each typically being present in an amount of from 0.01 to 25% by weight of the cosmetic composition, in particular from about 0.1 to 5% by weight of the cosmetic composition.

The compositions may include liposomes. The liposomes may comprise other additives or substances and/or may be modified to more specifically reach or remain at a site following administration.

In one embodiment, the composition of the invention may have a pH between about 1 and about 8. In certain embodiments, the pH of the composition will be acidic, i.e., less than 7.0., and preferably will be between about 2 and about 7, more preferably between about 3.5 and about 5.5.

In some embodiments, the invention provides methods for improving the aesthetic appearance of skin, preventing, reducing or treating one or more dermatological signs of skin aging, and/or treating aging skin. The methods comprise topically applying a cosmetic composition of the invention, preferably in a cosmetically acceptable vehicle, over the affected area for a period of time sufficient to reduce, ameliorate, reverse or prevent one or more dermatological signs of aging. In some embodiments, the compositions comprise upregulators and/or downregulators of one or more of the genes identified in Table 1. This method is particularly useful for treating signs of skin photoaging and intrinsic aging, such as for decreasing or increasing skin pigmentation. In some embodiments, the foregoing genes will be genes other than those selected from the group consisting of: tyrosinase, tyrosinase-related protein-1, premelanosome protein, melan-A endothelin receptor type B, keratin 6B, genes relating to melanogenesis, genes relating to inflammation, and genes relating to fatty acid metabolism.

Generally, the improvement in the condition and/or aesthetic appearance is selected from the group consisting of: reducing dermatological signs of chronological aging, photo-aging, hormonal aging, and/or actinic aging; preventing, reducing, and/or treating hyperpigmentation or hypopigmentation; preventing and/or reducing the appearance of lines and/or wrinkles; reducing the noticeability of facial lines and wrinkles, facial wrinkles on the cheeks, forehead, perpendicular wrinkles between the eyes, horizontal wrinkles above the eyes, and around the mouth, marionette lines, and particularly deep wrinkles or creases; preventing, reducing, and/or diminishing the appearance and/or depth of lines and/or wrinkles; improving the appearance of suborbital lines and/or periorbital lines; reducing the appearance of crow's feet; rejuvenating and/or revitalizing skin, particularly aging skin; reducing skin fragility; preventing and/or reversing of loss of glycosaminoglycans and/or collagen; ameliorating the effects of estrogen imbalance; preventing skin atrophy; minimizing skin discoloration; improving skin tone, radiance, clarity and/or tautness; preventing, reducing, and/or ameliorating skin sagging; improving skin firmness, plumpness, suppleness and/or softness; improving procollagen and/or collagen production; improving skin texture and/or promoting retexturization; improving skin barrier repair and/or function; improving the appearance of skin contours; restoring skin luster and/or brightness; minimizing dermatological signs of fatigue and/or stress; resisting environmental stress; replenishing ingredients in the skin decreased by aging and/or menopause; improving communication among skin cells; increasing cell proliferation and/or multiplication; increasing skin cell metabolism decreased by aging and/or menopause; retarding cellular aging; improving skin moisturization; enhancing skin thickness; increasing skin elasticity and/or resiliency; enhancing exfoliation; improving microcirculation; decreasing and/or preventing cellulite formation; and any combinations thereof.

The compositions may be applied to the skin for a period of time sufficient to diminish the appearance of melanin in the skin. The cosmetic composition will typically be applied to the skin for at least one, two, or three times daily for as long as is necessary to achieve desired anti-aging results. The treatment regimen may comprise at least daily application for at least one week, at least two weeks, at least four weeks, at least eight weeks, or at least twelve weeks. Chronic treatment regimens are also contemplated.

The aesthetic improvement of human skin (i.e., improvement in one or more dermatological signs of aging) achieved with the compositions of the invention may include, without limitation, one or more of the following:
(a) treatment, reduction, and/or prevention of fine lines or wrinkles;
(b) reduction of skin pore size;
(c) improvement in skin thickness, plumpness, and/or tautness;
(d) improvement in skin smoothness, suppleness and/or softness;
(e) improvement in skin tone, radiance, and/or clarity;
(f) improvement in procollagen, and/or collagen production;
(g) improvement in maintenance and remodeling of elastin;
(h) improvement in skin texture and/or promotion of retexturization;
(i) improvement in skin barrier repair and/or function;
(j) improvement in appearance of skin contours;
(k) restoration of skin luster and/or brightness;
(l) replenishment of essential nutrients and/or constituents in the skin;
(m) improvement of skin appearance decreased by aging and/or menopause;
(n) improvement in skin moisturization;
(o) increase in skin elasticity and/or resiliency;
(p) treatment, reduction, and/or prevention of skin sagging;
(q) improvement in skin firmness; and
(r) reduction of pigment spots and/or mottled skin; and
(s) improvement of optical properties of skin by light diffraction or reflection.

In practice, the compositions of the invention are applied to skin in need of treatment. Skin in need of treatment typically includes skin that suffers from a deficiency or loss in any of the foregoing attributes or which would otherwise benefit from improvement in any of the foregoing skin attributes.

In some embodiments, compositions and methods are provided for the prevention, treatment, and/or amelioration of skin discoloration, such as hyperpigmentation or hypopigmentation in human skin. Compositions of the invention may be applied to skin in need of treatment, by which is meant skin having, for example, hyperpigmentation or hypopigmentation. Hyperpigmentation includes any coloration of an individual's skin that is darker than desired by the individual. Such unwanted pigmentation may also be called discoloration. Hyperpigmented areas of the skin include areas of discrete or mottled hyper-pigmentation. Areas of discrete hyperpigmentation can be distinct, uniform areas of darker color and may appear as brown spots or freckles on the skin, including marks commonly called pigment spots or “age spots.” Hyperpigmentation also refers to senile lentigos or solar lentigos. Areas of mottled hyperpigmentation of the skin can be dark blotches that are larger and more irregular in size and shape than areas of discrete pigmentation. Areas of hyperpigmentation also include areas of tanned skin, e.g., skin tanned due to UV exposure.

Skin hyperpigmentation also includes scarring, or discoloration due to skin injury, including lacerations, burns, sunburn, acne, or other dermatological conditions. For example, skin hyperpigmented areas include melasmic/cholasmic patches resulting from melasma/cholasma, a common skin disorder involving facial skin discoloration. Melasmic (or chloasmic) patches may appear as dark brown, irregular patches on the face, upper cheeks, nose, lips, upper lip, and forehead, for example. Skin hyperpigmentation may also include areas under an individual's eyes that are darker than desired by the individual, commonly referred to as “under eye dark circles” or “dark circles.” Dark circles are usually round, uniform areas of pigmentation beneath each eye, which may be caused by heredity, allergies, tiredness, or other causes.

In some embodiments, methods of increasing skin pigmentation are provided, comprising topically administering to skin in need thereof an effective amount of a composition comprising a topically acceptable vehicle and an upregulator of any one or more of the genes in Table 1 (or any homolog, fragment, or marker of those genes) for a time sufficient to achieve a reduction in skin pigmentation.

In other embodiments, methods of decreasing skin pigmentation are provided, comprising topically administering to skin in need thereof an effective amount of a composition comprising a topically acceptable vehicle and a downregulator of any one or more of the genes in Table 1 (or any homolog, fragment, or marker of those genes) for a time sufficient to achieve an increase in skin pigmentation.

In other embodiments, methods of treating skin are provided, comprising topically administering to skin in need thereof an effective amount of a composition comprising a topically acceptable vehicle and a modulator (e.g., upregulator or downregulator) of any one or more of the genes in Table 1 (or any homolog, fragment, or marker of those genes) for a time sufficient to improve the aesthetic appearance of skin or one or more dermatological signs of aging.

Methods of treating and/or preventing hyperpigmented or hypopigmented skin are also provided.

Treating hyperpigmentation or hyperpigmented skin refers to eradicating, reducing, ameliorating, or reversing one or more of the unwanted features associated with hyperpigmentation, such as producing a perceptible lightening of the skin in the affected area. Lightening hyperpigmented areas of the skin may be desirable, in one embodiment, in diminishing age spots; lightening a suntan; evening or optimizing skin tones, e.g., in areas of mottled hyper-pigmentation; in treating melasmic and chloasmic patches, freckles, after-burn scars, and post-injury hyperpigmentation. Preventing hyperpigmentation or hyperpigmented skin refers to affording skin, not yet affected by hyperpigmentation, a benefit that serves to avoid, delay, forestall, or minimize one or more unwanted features associated with skin hyperpigmentation, such as reducing the darkness or size of hyperpigmented areas that eventually develop.

Treating hypopigmentation or hypopigmented skin refers to eradicating, reducing, ameliorating, or reversing one or more of the unwanted features associated with hypopigmentation, such as producing a perceptible darkening of the skin in the affected area. Darkening hypopigmented areas of the skin may be desirable, in one embodiment, in individuals that have pale patches of skin, or patches of depigmented skin associated with vitiligo or albinism. Preventing hypopigmentation or hypopigmented skin refers to affording skin, not yet affected by hypopigmentation, a benefit that serves to avoid, delay, forestall, or minimize one or more unwanted features associated with skin hypopigmentation, such as reducing the lightness or size of hypopigmented areas that eventually develop.

The compositions of the invention may be applied directly to the area of the skin that is hyperpigmented (e.g., age spots, freckles, suntan, dark circles under the eyes) or hypopigmented (e.g., depigmented patches or pale patches).

In other embodiments the compositions and methods of the invention are directed to the prevention, treatment, and/or amelioration of fine lines and/or wrinkles in the skin. In this case, the compositions are applied to skin in need of treatment, by which is meant skin having wrinkles and/or fine lines. In some embodiments, the compositions are applied directly to the fine lines and/or wrinkles. The compositions and methods are suitable for treating fine lines and/or wrinkles on any surface of the skin, including without limitation, the skin of the face, neck, and/or hands.

In other embodiments, the compositions and methods of the invention are directed to the prevention, treatment, and/or amelioration of blemishes or acne in human skin. In this case, the compositions are applied to skin in need of treatment, by which is meant skin having a blemish or acne. The compositions may be applied directly to the blemish or acne.

In other preferred embodiments, the compositions and methods of the invention are directed to promoting exfoliation of human skin. In this case, the compositions are applied to skin in need of treatment, by which is meant skin in need of exfoliation. The compositions may be applied directly to the area of skin in need of exfoliation.

In other embodiments, compositions may be used to treat, prevent, or ameliorate skin pigmentation, dandruff, seborrheic dermatitis, ringworm infection, psoriasis, calluses, ichthyosis, and warts.

The compositions are topically applied to an “individual in need thereof,” by which is meant an individual that stands to benefits from reducing visible signs of skin damage or aging (e.g., hyperpigmentation such as age spots, or hypopigmentation, such as pale or depigmented patches). In a specific embodiment, the active ingredient is provided in a pharmaceutically, physiologically, cosmetically, and dermatologically-acceptable vehicle, diluent, or carrier, where the composition is topically applied to an affected area of skin and left to remain on the affected area in an amount effective for improving the condition and aesthetic appearance of skin, such as improving hyperpigmentation or hypopigmentation.

The effect of a composition on the formation or appearance of age spots, freckles, fine lines and wrinkles, of a blemish or of acne, etc. can be evaluated qualitatively, e.g., by visual inspection, or quantitatively, e.g., by microscopic or computer assisted measurements of wrinkle morphology (e.g., the number, depth, length, area, volume and/or width of wrinkles per unit area of skin), etc.

It is also contemplated that the compositions of the invention will be useful for treating thin skin by topically applying the composition to thin skin of an individual in need thereof. “Thin skin” is intended to include skin that is thinned due to chronological aging, menopause, or photo-damage. In some embodiments, the treatment is for thin skin in men, whereas other embodiments treat thin skin in women, pre-menopausal or post-menopausal, as it is believed that skin thins differently with age in men and women, and in particular in women at different stages of life.

The methods of the invention may be employed prophylactically to forestall aging including in patients that have not manifested signs of skin aging, most commonly in male or female individuals under 25 years of age, under 35 years of age, or under 45 years of age. The methods may also reverse or treat signs of aging once manifested as is common in male or female individuals over 25 years of age, over 35 years of age, over 45 years of age, over 55 years of age, or over 65 years of age. The methods of the invention may also be used in male or female individuals either under 25 years of age or 25 years of age or older, to prevent, reverse, or treat acne, blemishes, hyperpigmentation, to improve the aesthetic appearance of skin, or to promote exfoliation of skin.

The following example is meant to demonstrate certain aspects of the invention in a non-limiting fashion.

### EXAMPLE

### Example 1

In order to identify a unique set of genes that modulate skin pigmentation, gene array analyses were conducted on mRNA isolated from human skin age spots, human skin adjacent to skin having age spots, and human skin that had been protected from the sun, and had no age spots.

Skin samples for the analyses were obtained from twelve Caucasian postmenopausal females between 55 and 65 years of age, with Fitzpatrick skin types II-III. A dermatologist examined the skin pigmentation of subjects, and with the aid of dermoscopy determined whether or not a subject had age spots (senile lentigo lesions). From each subject, 2-mm punch biopsies were taken from three age spots on the skin, on the dorsal aspect of the forearm (the “Lesional Group”), from three peri-lesional areas of skin adjacent to the age spots on the dorsal aspect of the forearm (the “Peri-Lesional Group), and from from a sun-protected area of the buttock that had no age spots (the “Protected Group”). Collected tissue was fixed in RNAlater and then analyzed for gene expression.

RNA was extracted from all tissue biopsy samples using the RNeasy method (Qiagen). Briefly, tissue was homogenized and lysed in RLT buffer (with beta-mercaptoethanol) using Lysing Matrix D tubes (MP Biomedicals LLC). Homogenates were transferred to collection tubes which were then loaded into a QIAcube automated sample prep instrument to perform the remainder of the RNeasy extraction. The resulting RNA was quantitated by spectrophotometry using a NanoDrop ND-8000 (Thermo), and was evaluated for integrity using the RNA 6000 Nano Assay on a Bioanalyzer 2100 (Agilent). Purified RNA samples were stored at −80° C.

RNA samples were converted into labelled target antisense RNA (cRNA) using the Single-Round RNA Amplification and Biotin Labelling System (Enzo Biochem). Briefly, 1 μg of total RNA was converted into double stranded cDNA via reverse transcription using an oligo-d(T) primer-adaptor. This cDNA was purified and used as a template for in vitro transcription using T7 RNA polymerase and biotinylated ribonucleotides. The resulting cRNA was purified using magnetic beads and quantitated using spectrophotometry. Next, 11 μg of purified cRNA was fragmented using a 5× Fragmentation buffer, and then a hybridization cocktail was prepared and added to the fragmentation product using the Hybridization, Wash and Stain kit (Affymetrix). This was applied to Human U133 Plus 2.0 gene arrays, and incubated at 45° C. for 16 hours. Following hybridization, arrays were washed and stained using standard Affymetrix procedures before scanning on the Affymetrix GeneChip Scanner. Data were extracted using Expression Console.

Gene expression was examined to determine which genes showed at least a 1.3 fold change in gene expression levels (whether upregulated or downregulated) in the Lesional Group relative to the Protected Group, and 482 genes fit these criteria. Gene expression was also examined to determine which genes showed at least a 1.2 fold change in gene expression (whether upregulated or downregulated) in the Lesional Group relative to the Peri-Lesional Group, and 118 genes fit these criteria. A unique set of 52 genes was modulated in both sets of comparisons. These genes, their symbols, names, and their associated accession numbers (according to the National Center for Biotechnology Information (NCBI) database) are identified below in Table 1, along with the location of the gene, and the fold-change quantified for each comparison. A negative number indicates that the gene was downregulated, and a positive number indicates that the gene was upregulated.

TABLE 1

| | | | | | Fold |
|---|---|---|---|---|---|
| | | | | Fold | Change |
| | NCBI | | | Change | (Peri- |
| | RefSeq | | | (Protected | Lesional |
| NCBI | Accession | NCBI Entrez Gene | | vs. | vs. |
| Symbol | No. | Name | Location | Lesional) | Lesional) |
| CYP39A1 | NM_016593 | Homo sapiens | Cytoplasm | −2.027 | −1.781 |
| | | cytochrome P450, | | | |
| | | family 39, subfamily A, | | | |
| | | polypeptide 1, | | | |
| | | transcript variant 1, | | | |
| | | mRNA | | | |
| ACTC1 | NM_005159 | Homo sapiens actin, | Cytoplasm | −1.421 | −1.423 |
| | | alpha, cardiac muscle | | | |
| | | 1, mRNA | | | |
| POSTN | NM_006475 | Homo sapiens | Extracellular | −1.526 | −1.392 |
| | | periostin, osteoblast | Space | | |
| | | specific factor, | | | |
| | | transcript variant 1, | | | |
| | | mRNA | | | |
| SLITRK6 | NM_032229 | Homo sapiens SLIT | Plasma | −1.367 | −1.373 |
| | | and NTRK-like family, | Membrane | | |
| | | member 6, mRNA | | | |
| RPS6KA6 | NM_014496 | Homo sapiens | Cytoplasm | −1.353 | −1.370 |
| | | ribosomal protein S6 | | | |
| | | kinase, 90 kDa, | | | |
| | | polypeptide 6, mRNA | | | |
| F2R | NM_001992 | Homo sapiens | Plasma | −1.506 | −1.362 |
| | | coagulation factor II | Membrane | | |
| | | (thrombin) receptor, | | | |
| | | mRNA | | | |
| ACTC1 | NM_005159 | Homo sapiens actin, | Cytoplasm | −1.421 | −1.423 |
| | | alpha, cardiac muscle | | | |
| | | 1, mRNA | | | |
| FLRT3 | NM_198391 | Homo sapiens | Plasma | −1.407 | −1.355 |
| | | fibronectin leucine rich | Membrane | | |
| | | transmembrane protein | | | |
| | | 3, transcript variant 2, | | | |
| | | mRNA | | | |
| RNF180 | NM_001113561 | Homo sapiens ring | Cytoplasm | −1.615 | −1.352 |
| | | finger protein 180, | | | |
| | | transcript variant 1, | | | |
| | | mRNA | | | |
| NMNAT3 | NM_178177 | Homo sapiens | Cytoplasm | −1.437 | −1.302 |
| | | nicotinamide | | | |
| | | nucleotide | | | |
| | | adenylyltransferase 3, | | | |
| | | transcript variant 1, | | | |
| | | mRNA | | | |
| OR2T2 | NM_001004136 | Homo sapiens olfactory | Plasma | −1.513 | −1.297 |
| | | receptor, family 2, | Membrane | | |
| | | subfamily T, member | | | |
| | | 2, mRNA | | | |
| ZNF204P | NR_002722 | Homo sapiens zinc | Other | −1.414 | −1.280 |
| | | finger protein 204, | | | |
| | | pseudogene, transcript | | | |
| | | variant 1, non-coding | | | |
| | | RNA | | | |
| SLC38A4 | NM_018018 | Homo sapiens solute | Plasma | −1.326 | −1.260 |
| | | carrier family 38, | Membrane | | |
| | | member 4, transcript | | | |
| | | variant 1, mRNA | | | |
| SLC25A27 | NM_004277 | Homo sapiens solute | Cytoplasm | −1.317 | −1.243 |
| | | carrier family 25, | | | |
| | | member 27, transcript | | | |
| | | variant 1, mRNA | | | |
| KIF21A | NM_001173464 | Homo sapiens kinesin | Cytoplasm | −1.315 | −1.239 |
| | | family member 21A, | | | |
| | | transcript variant 1, | | | |
| | | mRNA | | | |
| SPINK1 | NM_003122 | Homo sapiens serine | Extracellular | −1.356 | −1.219 |
| | | peptidase inhibitor, | Space | | |
| | | Kazal type 1, mRNA | | | |
| MX2 | NM_002463 | Homo sapiens MX | Nucleus | 1.399 | 1.203 |
| | | dynamin-like GTPase | | | |
| | | 2, mRNA | | | |
| C10orf90 | NM_001004298 | Homo sapiens | Cytoplasm | 1.689 | 1.215 |
| | | chromosome 10 open | | | |
| | | reading frame 90, | | | |
| | | mRNA | | | |
| MYO5A | NM_000259 | Homo sapiens myosin | Cytoplasm | 1.472 | 1.215 |
| | | VA (heavy chain 12, | | | |
| | | myoxin), transcript | | | |
| | | variant 1, mRNA | | | |
| PLXNC1 | NM_005761 | Homo sapiens plexin | Plasma | 1.614 | 1.226 |
| | | C1, transcript variant 1, | Membrane | | |
| | | mRNA | | | |
| MCOLN3 | NM_018298 | Homo sapiens | Plasma | 1.916 | 1.240 |
| | | mucolipin 3, transcript | Membrane | | |
| | | variant 1, mRNA | | | |
| OCA2 | NM_000275 | Homo sapiens | Plasma | 1.605 | 1.241 |
| | | oculocutaneous | Membrane | | |
| | | albinism II, transcript | | | |
| | | variant 1, mRNA | | | |
| MLANA | NM_005511 | Homo sapiens melan- | Plasma | 2.125 | 1.244 |
| | | A, mRNA | Membrane | | |
| OAS2 | NM_002535 | Homo sapiens 2′-5′- | Cytoplasm | 1.517 | 1.250 |
| | | oligoadenylate | | | |
| | | synthetase 2, | | | |
| | | 69/71 kDa, transcript | | | |
| | | variant 2, mRNA | | | |
| GPR143 | NM_000273 | Homo sapiens G | Plasma | 1.750 | 1.252 |
| | | protein-coupled | Membrane | | |
| | | receptor 143, mRNA | | | |
| PPAPDC1A | NM_001030059 | Homo sapiens | Plasma | 1.386 | 1.265 |
| | | phosphatidic acid | Membrane | | |
| | | phosphatase type 2 | | | |
| | | domain containing 1A, | | | |
| | | mRNA | | | |
| EMP3 | NM_001425 | Homo sapiens | Plasma | 1.568 | 1.276 |
| | | epithelial membrane | Membrane | | |
| | | protein 3, mRNA | | | |
| PAX3 | NM_181458 | Homo sapiens paired | Nucleus | 1.366 | 1.286 |
| | | box 3, transcript variant | | | |
| | | PAX3D, mRNA | | | |
| SLC1A4 | NM_003038 | Homo sapiens solute | Plasma | 1.508 | 1.287 |
| | | carrier family 1 | Membrane | | |
| | | (glutamate/neutral | | | |
| | | amino acid transporter), | | | |
| | | member 4, transcript | | | |
| | | variant 1, mRNA | | | |
| LINC00518 | NR_027793 | Homo sapiens long | Other | 1.695 | 1.288 |
| | | intergenic non-protein | | | |
| | | coding RNA 518, long | | | |
| | | non-coding RNA | | | |
| ENPP2 | NM_006209 | Homo sapiens | Plasma | 1.622 | 1.290 |
| | | ectonucleotide | Membrane | | |
| | | pyrophosphatase/phosphodiesterase | | | |
| | | 2, | | | |
| | | transcript variant 1, | | | |
| | | mRNA | | | |
| NREP | NM_004772 | Homo sapiens neuronal | Cytoplasm | 1.454 | 1.292 |
| | | regeneration related | | | |
| | | protein, transcript | | | |
| | | variant 1, mRNA | | | |
| CD24 | NM_013230 | Homo sapiens CD24 | Plasma | 1.556 | 1.293 |
| | | molecule, transcript | Membrane | | |
| | | variant 1, mRNA | | | |
| PCDH7 | NM_032456 | Homo sapiens | Plasma | 1.409 | 1.294 |
| | | protocadherin 7, | Membrane | | |
| | | transcript variant b, | | | |
| | | mRNA | | | |
| SERPINB12 | NM_080474 | Homo sapiens serpin | Cytoplasm | 1.346 | 1.312 |
| | | peptidase inhibitor, | | | |
| | | clade B (ovalbumin), | | | |
| | | member 12, mRNA | | | |
| TRPM1 | NM_002420 | Homo sapiens transient | Plasma | 2.368 | 1.345 |
| | | receptor potential | Membrane | | |
| | | cation channel, | | | |
| | | subfamily M, member | | | |
| | | 1, transcript variant 2, | | | |
| | | mRNA | | | |
| EDNRB | NM_001122659 | Homo sapiens | Plasma | 2.344 | 1.350 |
| | | endothelin receptor | Membrane | | |
| | | type B, transcript | | | |
| | | variant 3, mRNA | | | |
| WDR63 | NM_145172 | Homo sapiens WD | Other | 1.568 | 1.365 |
| | | repeat domain 63, | | | |
| | | transcript variant 1, | | | |
| | | mRNA | | | |
| TYR | NM_000372 | Homo sapiens | Cytoplasm | 2.661 | 1.399 |
| | | tyrosinase, mRNA | | | |
| TLL1 | NM_012464 | Homo sapiens tolloid- | Extracellular | 1.705 | 1.428 |
| | | like 1, transcript variant | Space | | |
| | | 1, mRNA | | | |
| TYRP1 | NM_000550 | Homo sapiens | Cytoplasm | 3.564 | 1.446 |
| | | tyrosinase-related | | | |
| | | protein 1, mRNA | | | |
| PMEL | NM_006928 | Homo sapiens | Plasma | 3.768 | 1.448 |
| | | premelanosome | Membrane | | |
| | | protein, transcript | | | |
| | | variant 3, mRNA | | | |
| HMCN1 | NM_031935 | Homo sapiens | Extracellular | 1.748 | 1.500 |
| | | hemicentin 1, mRNA | Space | | |
| HOXD11 | NM_021192 | Homo sapiens | Nucleus | 1.822 | 1.513 |
| | | homeobox D11, mRNA | | | |
| KRT6B | NM_005555 | Homo sapiens keratin | Cytoplasm | 1.779 | 1.521 |
| | | 6B, mRNA | | | |
| IGFBP3 | NM_001013398 | Homo sapiens insulin- | Extracellular | 1.342 | 1.532 |
| | | like growth factor | Space | | |
| | | binding protein 3, | | | |
| | | transcript variant 1, | | | |
| | | mRNA | | | |
| FRAS1 | NM_025074 | Homo sapiens Fraser | Extracellular | 1.391 | 1.542 |
| | | extracellular matrix | Space | | |
| | | complex subunit 1, | | | |
| | | transcript variant 1, | | | |
| | | mRNA | | | |
| FREM2 | NM_207361 | Homo sapiens FRAS1 | Extracellular | 1.435 | 1.548 |
| | | related extracellular | Space | | |
| | | matrix protein 2, | | | |
| | | mRNA | | | |
| SPRR1A | NM_005987 | Homo sapiens small | Plasma | 1.633 | 1.642 |
| | | proline-rich protein 1A, | Membrane | | |
| | | transcript variant 2, | | | |
| | | mRNA | | | |
| FSHR | NM_000145 | Homo sapiens follicle | Plasma | 1.618 | 1.669 |
| | | stimulating hormone | Membrane | | |
| | | receptor, transcript | | | |
| | | variant 1, mRNA | | | |
| ATP12A | NM_001185085 | Homo sapiens ATPase, | Plasma | 1.637 | 1.687 |
| | | H+/K+ transporting, | Membrane | | |
| | | nongastric, alpha | | | |
| | | polypeptide, transcript | | | |
| | | variant 1, mRNA | | | |
| HOXD10 | NM_002148 | Homo sapiens | Nucleus | 3.611 | 2.114 |
| | | homeobox D10, mRNA | | | |

It will be understood that in the event of any inconsistency between the gene symbol, accession number, and gene name in Table 1, all such nucleic acids will be considered as embraced by the invention. For example, in the event of a discrepancy in the accession number and the gene name provided, both the gene corresponding to the accession number and the gene corresponding to the gene name will be considered within the scope of the invention.

Following treatment of skin with a candidate substance, modulation of expression levels of any two or more of the genes identified in Table 1 (or expression of any homolog, fragment or marker of those genes) indicates that the candidate substance may modulate skin pigmentation, such as, for example, by reducing skin pigmentation, or by increasing skin pigmentation.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

### EMBODIMENTS

1. A method of screening for modulators of skin pigmentation comprising:
(a) contacting a human skin cell with a candidate substance; and
(b) determining whether expression of at least two genes is modulated in said skin cell, said genes being selected from the group consisting of: MX dynamin-like GTPase 2; chromosome 10 open reading frame 90; myosin VA (heavy chain 12, myoxin); plexin C1; mucolipin 3; oculocutaneous albinism II; melan-A; 2′-5′-oligoadenylate synthetase 2, 69/71 kDa; G protein-coupled receptor 143; phosphatidic acid phosphatase type 2 domain containing 1A; epithelial membrane protein 3; paired box 3; solute carrier family 1 (glutamate/neutral amino acid transporter), member 4; long intergenic non-protein coding RNA 518; ectonucleotide pyrophosphatase/phosphodiesterase 2; neuronal regeneration related protein; CD24 molecule; protocadherin 7; serpin peptidase inhibitor, clade B (ovalbumin), member 12; transient receptor potential cation channel, subfamily M, member 1; endothelin receptor type B; WD repeat domain 63; tolloid-like 1; tyrosinase-related protein 1; premelanosome protein; hemicentin 1; homeobox D11; keratin 6B; insulin-like growth factor binding protein 3; Fraser extracellular matrix complex subunit 1; FRAS1 related extracellular matrix protein 2; small proline-rich protein 1A; follicle stimulating hormone receptor; ATPase, H+/K+ transporting, nongastric, alpha polypeptide; homeobox D10; cytochrome P450, family 39, subfamily A, polypeptide 1; actin, alpha, cardiac muscle 1; periostin, osteoblast specific factor; SLIT and NTRK-like family, member 6; ribosomal protein S6 kinase, 90 kDa, polypeptide 6; coagulation factor II (thrombin) receptor; actin, alpha, cardiac muscle 1; fibronectin leucine rich transmembrane protein 3; ring finger protein 180; nicotinamide nucleotide adenylyltransferase 3; olfactory receptor, family 2, subfamily T, member 2; zinc finger protein 204, pseudogene; solute carrier family 38, member 4; solute carrier family 25, member 27; kinesin family member 21A; serine peptidase inhibitor, and Kazal type 1;
   wherein modulation of said at least two genes indicates that said candidate substance modulates skin pigmentation.

2. The method according to claim 1, wherein said skin cell is a fibroblast or a keratinocyte.

3. The method according to claim 1, wherein said modulation comprises upregulation or downregulation of said two or more genes.

4. The method according to claim 1, wherein said step of determining further comprises obtaining a skin cell, and extracting nucleic acids from said skin cell.

5. The method according to claim 1, wherein said step of determining further comprises quantifying said nucleic acids extracted from said skin cell.

6. The method according to claim 1, further comprising determining whether expression of said two or more genes is modulated in a skin cell that has not been contacted with said candidate substance.

7. The method according to claim 1, wherein said modulation comprises between about a 1.2-fold change and about a 10-fold change in gene expression relative to expression of the same genes in a skin cell that has not been contacted with said candidate substance.

8. The method according to claim 1, wherein expression of at least three genes is modulated in said skin cell.

9. The method according to claim 1, wherein expression of at least one gene is upregulated and expression of at least one gene is downregulated in said skin cell.

10. A method of reducing skin pigmentation, comprising topically applying to human skin in need thereof a cosmetic composition comprising an effective amount of a downregulator of one or more of the genes selected from the group consisting of: MX dynamin-like GTPase 2; chromosome 10 open reading frame 90; myosin VA (heavy chain 12, myoxin); plexin C1; mucolipin 3; oculocutaneous albinism II; melan-A; 2′-5′-oligoadenylate synthetase 2, 69/71 kDa; G protein-coupled receptor 143; phosphatidic acid phosphatase type 2 domain containing 1A; epithelial membrane protein 3; paired box 3; solute carrier family 1 (glutamate/neutral amino acid transporter), member 4; long intergenic non-protein coding RNA 518; ectonucleotide pyrophosphatase/phosphodiesterase 2; neuronal regeneration related protein; CD24 molecule; protocadherin 7; serpin peptidase inhibitor, clade B (ovalbumin), member 12; transient receptor potential cation channel, subfamily M, member 1; endothelin receptor type B; WD repeat domain 63; tolloid-like 1; tyrosinase-related protein 1; premelanosome protein; hemicentin 1; homeobox D11; keratin 6B; insulin-like growth factor binding protein 3; Fraser extracellular matrix complex subunit 1; FRAS1 related extracellular matrix protein 2; small proline-rich protein 1A; follicle stimulating hormone receptor; ATPase, H+/K+ transporting, nongastric, alpha polypeptide; and homeobox D10;

wherein said downregulator of said one or more genes is present in an amount sufficient to decrease pigmentation in said skin.

11. The method according to claim 1, wherein at least one of the genes downregulated is selected from the group consisting of: homeobox D10; transient receptor potential cation channel, subfamily M, member 1; and mucolipin 3; homeobox D11; hemicentin 1; G protein-coupled receptor 143; and long intergenic non-protein coding RNA 518.

12. A method of reducing skin pigmentation, comprising topically applying to human skin in need thereof a cosmetic composition comprising an effective amount of an upregulator of one or more of the genes selected from the group consisting of: cytochrome P450, family 39, subfamily A, polypeptide 1; actin, alpha, cardiac muscle 1; periostin, osteoblast specific factor; SLIT and NTRK-like family, member 6; ribosomal protein S6 kinase, 90 kDa, polypeptide 6; coagulation factor II (thrombin) receptor; actin, alpha, cardiac muscle 1; fibronectin leucine rich transmembrane protein 3; ring finger protein 180; nicotinamide nucleotide adenylyltransferase 3; olfactory receptor, family 2, subfamily T, member 2; zinc finger protein 204, pseudogene; solute carrier family 38, member 4; solute carrier family 25, member 27; kinesin family member 21A; serine peptidase inhibitor, and Kazal type 1;

wherein said upregulator of said one or more genes is present in an amount sufficient to decrease pigmentation in said skin.

13. The method according to claims 10-12, wherein the composition is applied to skin of the face, neck, arms, or legs.

14. The method according to claim 10 or 12, wherein the composition is applied to an area of skin having hyperpigmentation.

15. The method according to claims 10-12, wherein the composition is applied to the skin for at least one week.

16. A cosmetic composition for topical application comprising, in a cosmetically acceptable vehicle, a downregulator of one or more of the genes selected from the group consisting of: MX dynamin-like GTPase 2; chromosome 10 open reading frame 90; myosin VA (heavy chain 12, myoxin); plexin C1; mucolipin 3; oculocutaneous albinism II; melan-A; 2′-5′-oligoadenylate synthetase 2, 69/71 kDa; G protein-coupled receptor 143; phosphatidic acid phosphatase type 2 domain containing 1A; epithelial membrane protein 3; paired box 3; solute carrier family 1 (glutamate/neutral amino acid transporter), member 4; long intergenic non-protein coding RNA 518; ectonucleotide pyrophosphatase/phosphodiesterase 2; neuronal regeneration related protein; CD24 molecule; protocadherin 7; serpin peptidase inhibitor, clade B (ovalbumin), member 12; transient receptor potential cation channel, subfamily M, member 1; endothelin receptor type B; WD repeat domain 63; tolloid-like 1; tyrosinase-related protein 1; premelanosome protein; hemicentin 1; homeobox D11; keratin 6B; insulin-like growth factor binding protein 3; Fraser extracellular matrix complex subunit 1; FRAS1 related extracellular matrix protein 2; small proline-rich protein 1A; follicle stimulating hormone receptor; ATPase, H+/K+ transporting, nongastric, alpha polypeptide; and homeobox D10.

17. A cosmetic composition for topical application comprising, in a cosmetically acceptable vehicle, an upregulator of one or more of the genes selected from the group consisting of: cytochrome P450, family 39, subfamily A, polypeptide 1; actin, alpha, cardiac muscle 1; periostin, osteoblast specific factor; SLIT and NTRK-like family, member 6; ribosomal protein S6 kinase, 90 kDa, polypeptide 6; coagulation factor II (thrombin) receptor; actin, alpha, cardiac muscle 1; fibronectin leucine rich transmembrane protein 3; ring finger protein 180; nicotinamide nucleotide adenylyltransferase 3; olfactory receptor, family 2, subfamily T, member 2; zinc finger protein 204, pseudogene; solute carrier family 38, member 4; solute carrier family 25, member 27; kinesin family member 21A; serine peptidase inhibitor, and Kazal type 1.

18. The cosmetic composition according to claim 16 or 17, further comprising a cosmetic ingredient selected from the group consisting of a botanical extract, film forming polymer, a thickener, a pH adjuster, a preservative, an emulsifier, a gelling agent, an antioxidant, an emollient, a humectant, a fragrance, and a colorant.

19. A method for improving the aesthetic appearance of human skin comprising topically applying to an area of the skin in need thereof a composition according to claim 16 or 17.

20. The method according to claim 19, wherein said aesthetic improvement of said human skin is selected from the group consisting of: reduction of hyperpigmentation, reduction of age spots, reduction of mottled skin, and reduction in suntan.

21. A method for increasing skin pigmentation, comprising topically applying to human skin in need thereof a cosmetic composition comprising an effective amount of an upregulator of one or more of the genes selected from the group consisting of: MX dynamin-like GTPase 2; chromosome 10 open reading frame 90; myosin VA (heavy chain 12, myoxin); plexin C1; mucolipin 3; oculocutaneous albinism II; melan-A; 2′-5′-oligoadenylate synthetase 2, 69/71 kDa; G protein-coupled receptor 143; phosphatidic acid phosphatase type 2 domain containing 1A; epithelial membrane protein 3; paired box 3; solute carrier family 1 (glutamate/neutral amino acid transporter), member 4; long intergenic non-protein coding RNA 518; ectonucleotide pyrophosphatase/phosphodiesterase 2; neuronal regeneration related protein; CD24 molecule; protocadherin 7; serpin peptidase inhibitor, clade B (ovalbumin), member 12; transient receptor potential cation channel, subfamily M, member 1; endothelin receptor type B; WD repeat domain 63; tolloid-like 1; tyrosinase-related protein 1; premelanosome protein; hemicentin 1; homeobox D11; keratin 6B; insulin-like growth factor binding protein 3; Fraser extracellular matrix complex subunit 1; FRAS1 related extracellular matrix protein 2; small proline-rich protein 1A; follicle stimulating hormone receptor; ATPase, H+/K+ transporting, nongastric, alpha polypeptide; and homeobox D10;

wherein said upregulator of said one or more genes is present in an amount sufficient to increase pigmentation in said skin.

22. A method of increasing skin pigmentation, comprising topically applying to human skin in need thereof a cosmetic composition comprising an effective amount of a downregulator of one or more of the genes selected from the group consisting of: actin, alpha, cardiac muscle 1; periostin, osteoblast specific factor; SLIT and NTRK-like family, member 6; ribosomal protein S6 kinase, 90 kDa, polypeptide 6; coagulation factor II (thrombin) receptor; actin, alpha, cardiac muscle 1; fibronectin leucine rich transmembrane protein 3; ring finger protein 180; nicotinamide nucleotide adenylyltransferase 3; olfactory receptor, family 2, subfamily T, member 2; zinc finger protein 204, pseudogene; solute carrier family 38, member 4; solute carrier family 25, member 27; kinesin family member 21A; serine peptidase inhibitor, and Kazal type 1;

wherein said downregulator of said one or more genes is present in an amount sufficient to increase pigmentation in said skin.

23. A gene panel, wherein at least 50% of nucleic acids on said gene panel comprise oligonucleotides that hybridize with nucleic acids corresponding to genes selected from the group consisting of: cytochrome P450, family 39, subfamily A, polypeptide 1; actin, alpha, cardiac muscle 1; periostin, osteoblast specific factor; SLIT and NTRK-like family, member 6; ribosomal protein S6 kinase, 90 kDa, polypeptide 6; coagulation factor II (thrombin) receptor; actin, alpha, cardiac muscle 1; fibronectin leucine rich transmembrane protein 3; ring finger protein 180; nicotinamide nucleotide adenylyltransferase 3; olfactory receptor, family 2, subfamily T, member 2; zinc finger protein 204, pseudogene; solute carrier family 38, member 4; solute carrier family 25, member 27; kinesin family member 21A; serine peptidase inhibitor, Kazal type 1; MX dynamin-like GTPase 2; chromosome 10 open reading frame 90; myosin VA (heavy chain 12, myoxin); plexin C1; mucolipin 3; oculocutaneous albinism II; melan-A; 2′-5′-oligoadenylate synthetase 2, 69/71 kDa; G protein-coupled receptor 143; phosphatidic acid phosphatase type 2 domain containing 1A; epithelial membrane protein 3; paired box 3; solute carrier family 1 (glutamate/neutral amino acid transporter), member 4; long intergenic non-protein coding RNA 518; ectonucleotide pyrophosphatase/phosphodiesterase 2; neuronal regeneration related protein; CD24 molecule; protocadherin 7; serpin peptidase inhibitor, clade B (ovalbumin), member 12; transient receptor potential cation channel, subfamily M, member 1; endothelin receptor type B; WD repeat domain 63; tyrosinase; tolloid-like 1; tyrosinase-related protein 1; premelanosome protein; hemicentin 1; homeobox D11; keratin 6B; insulin-like growth factor binding protein 3; Fraser extracellular matrix complex subunit 1; FRAS1 related extracellular matrix protein 2; small proline-rich protein 1A; follicle stimulating hormone receptor; ATPase, H+/K+ transporting, nongastric, alpha polypeptide; and homeobox D10.

24. A gene panel, wherein at least 75% of nucleic acids on said gene panel comprise oligonucleotides that hybridize with nucleic acids corresponding to genes selected from the group consisting of: cytochrome P450, family 39, subfamily A, polypeptide 1; actin, alpha, cardiac muscle 1; periostin, osteoblast specific factor; SLIT and NTRK-like family, member 6; ribosomal protein S6 kinase, 90 kDa, polypeptide 6; coagulation factor II (thrombin) receptor; actin, alpha, cardiac muscle 1; fibronectin leucine rich transmembrane protein 3; ring finger protein 180; nicotinamide nucleotide adenylyltransferase 3; olfactory receptor, family 2, subfamily T, member 2; zinc finger protein 204, pseudogene; solute carrier family 38, member 4; solute carrier family 25, member 27; kinesin family member 21A; serine peptidase inhibitor, Kazal type 1; MX dynamin-like GTPase 2; chromosome 10 open reading frame 90; myosin VA (heavy chain 12, myoxin); plexin C1; mucolipin 3; oculocutaneous albinism II; melan-A; 2′-5′-oligoadenylate synthetase 2, 69/71 kDa; G protein-coupled receptor 143; phosphatidic acid phosphatase type 2 domain containing 1A; epithelial membrane protein 3; paired box 3; solute carrier family 1 (glutamate/neutral amino acid transporter), member 4; long intergenic non-protein coding RNA 518; ectonucleotide pyrophosphatase/phosphodiesterase 2; neuronal regeneration related protein; CD24 molecule; protocadherin 7; serpin peptidase inhibitor, clade B (ovalbumin), member 12; transient receptor potential cation channel, subfamily M, member 1; endothelin receptor type B; WD repeat domain 63; tyrosinase; tolloid-like 1; tyrosinase-related protein 1; premelanosome protein; hemicentin 1; homeobox D11; keratin 6B; insulin-like growth factor binding protein 3; Fraser extracellular matrix complex subunit 1; FRAS1 related extracellular matrix protein 2; small proline-rich protein 1A; follicle stimulating hormone receptor; ATPase, H+/K+ transporting, nongastric, alpha polypeptide; and homeobox D10.

25. A gene panel, wherein at least 90% of nucleic acids on said gene panel comprise oligonucleotides that hybridize with nucleic acids corresponding to genes selected from the group consisting of: cytochrome P450, family 39, subfamily A, polypeptide 1; actin, alpha, cardiac muscle 1; periostin, osteoblast specific factor; SLIT and NTRK-like family, member 6; ribosomal protein S6 kinase, 90 kDa, polypeptide 6; coagulation factor II (thrombin) receptor; actin, alpha, cardiac muscle 1; fibronectin leucine rich transmembrane protein 3; ring finger protein 180; nicotinamide nucleotide adenylyltransferase 3; olfactory receptor, family 2, subfamily T, member 2; zinc finger protein 204, pseudogene; solute carrier family 38, member 4; solute carrier family 25, member 27; kinesin family member 21A; serine peptidase inhibitor, Kazal type 1; MX dynamin-like GTPase 2; chromosome 10 open reading frame 90; myosin VA (heavy chain 12, myoxin); plexin C1; mucolipin 3; oculocutaneous albinism II; melan-A; 2′-5′-oligoadenylate synthetase 2, 69/71 kDa; G protein-coupled receptor 143; phosphatidic acid phosphatase type 2 domain containing 1A; epithelial membrane protein 3; paired box 3; solute carrier family 1 (glutamate/neutral amino acid transporter), member 4; long intergenic non-protein coding RNA 518; ectonucleotide pyrophosphatase/phosphodiesterase 2; neuronal regeneration related protein; CD24 molecule; protocadherin 7; serpin peptidase inhibitor, clade B (ovalbumin), member 12; transient receptor potential cation channel, subfamily M, member 1; endothelin receptor type B; WD repeat domain 63; tyrosinase; tolloid-like 1; tyrosinase-related protein 1; premelanosome protein; hemicentin 1; homeobox D11; keratin 6B; insulin-like growth factor binding protein 3; Fraser extracellular matrix complex subunit 1; FRAS1 related extracellular matrix protein 2; small proline-rich protein 1A; follicle stimulating hormone receptor; ATPase, H+/K+ transporting, nongastric, alpha polypeptide; and homeobox D10.

26. The gene panel according to any of claims 23-25, wherein said gene is cytochrome P450, family 39, subfamily A, polypeptide 1.

27. The gene panel according to any of claims 23-25, wherein said gene is actin, alpha, cardiac muscle 1.

28. The gene panel according to any of claims 23-25, wherein said gene is periostin, osteoblast specific factor.

29. The gene panel according to any of claims 23-25, wherein said gene is SLIT and NTRK-like family, member 6.

30. The gene panel according to any of claims 23-25, wherein said gene is ribosomal protein S6 kinase, 90 kDa, polypeptide 6.

31. The gene panel according to any of claims 23-25, wherein said gene is coagulation factor II (thrombin) receptor.

32. The gene panel according to any of claims 23-25, wherein said gene is cytochrome P450, family 39, subfamily A, polypeptide 1.

33. The gene panel according to any of claims 23-25, wherein said gene is actin, alpha, cardiac muscle 1.

34. The gene panel according to any of claims 23-25, wherein said gene is fibronectin leucine rich transmembrane protein 3.

35. The gene panel according to any of claims 23-25, wherein said gene is ring finger protein 180.

36. The gene panel according to any of claims 23-25, wherein said gene is nicotinamide nucleotide adenylyltransferase 3.

37. The gene panel according to any of claims 23-25, wherein said gene is olfactory receptor, family 2, subfamily T, member 2.

38. The gene panel according to any of claims 23-25, wherein said gene is zinc finger protein 204, pseudogene.

39. The gene panel according to any of claims 23-25, wherein said gene is solute carrier family 38, member 4.

40. The gene panel according to any of claims 23-25, wherein said gene is solute carrier family 25, member 27.

41. The gene panel according to any of claims 23-25, wherein said gene is kinesin family member 21A.

42. The gene panel according to any of claims 23-25, wherein said gene is serine peptidase inhibitor, Kazal type 1.

43. The gene panel according to any of claims 23-25, wherein said gene is MX dynamin-like GTPase 2.

44. The gene panel according to any of claims 23-25, wherein said gene is chromosome 10 open reading frame 90.

45. The gene panel according to any of claims 23-25, wherein said gene is myosin VA (heavy chain 12, myoxin).

46. The gene panel according to any of claims 23-25, wherein said gene is plexin C1.

47. The gene panel according to any of claims 23-25, wherein said gene is mucolipin 3.

48. The gene panel according to any of claims 23-25, wherein said gene is oculocutaneous albinism II.

49. The gene panel according to any of claims 23-25, wherein said gene is melan-A.

50. The gene panel according to any of claims 23-25, wherein said gene is 2′-5′-oligoadenylate synthetase 2, 69/71 kDa.

51. The gene panel according to any of claims 23-25, wherein said gene is G protein-coupled receptor 143.

52. The gene panel according to any of claims 23-25, wherein said gene is phosphatidic acid phosphatase type 2 domain containing 1A.

53. The gene panel according to any of claims 23-25, wherein said gene is epithelial membrane protein 3.

54. The gene panel according to any of claims 23-25, wherein said gene is paired box 3.

55. The gene panel according to any of claims 23-25, wherein said gene is solute carrier family 1 (glutamate/neutral amino acid transporter), member 4.

56. The gene panel according to any of claims 23-25, wherein said gene is long intergenic non-protein coding RNA 518.

57. The gene panel according to any of claims 23-25, wherein said gene is ectonucleotide pyrophosphatase/phosphodiesterase 2.

58. The gene panel according to any of claims 23-25, wherein said gene is neuronal regeneration related protein.

59. The gene panel according to any of claims 23-25, wherein said gene is CD24 molecule; protocadherin 7.

60. The gene panel according to any of claims 23-25, wherein said gene is serpin peptidase inhibitor, clade B (ovalbumin), member 12.

61. The gene panel according to any of claims 23-25, wherein said gene is transient receptor potential cation channel, subfamily M, member 1.

62. The gene panel according to any of claims 23-25, wherein said gene is endothelin receptor type B.

63. The gene panel according to any of claims 23-25, wherein said gene is WD repeat domain 63.

64. The gene panel according to any of claims 23-25, wherein said gene is tyrosinase.

65. The gene panel according to any of claims 23-25, wherein said gene is tolloid-like 1.

66. The gene panel according to any of claims 23-25, wherein said gene is tyrosinase-related protein 1.

67. The gene panel according to any of claims 23-25, wherein said gene is premelanosome protein.

68. The gene panel according to any of claims 23-25, wherein said gene is hemicentin 1.

69. The gene panel according to any of claims 23-25, wherein said gene is homeobox D11.

70. The gene panel according to any of claims 23-25, wherein said gene is keratin 6B.

71. The gene panel according to any of claims 23-25, wherein said gene is insulin-like growth factor binding protein 3.

72. The gene panel according to any of claims 23-25, wherein said gene is Fraser extracellular matrix complex subunit 1.

73. The gene panel according to any of claims 23-25, wherein said gene is FRAS1 related extracellular matrix protein 2.

74. The gene panel according to any of claims 23-25, wherein said gene is small proline-rich protein 1A.

75. The gene panel according to any of claims 23-25, wherein said gene is follicle stimulating hormone receptor.

76. The gene panel according to any of claims 23-25, wherein said gene is ATPase, H+/K+ transporting, nongastric, alpha polypeptide.

77. The gene panel according to any of claims 23-25, wherein said gene is homeobox D10.

78. A method of decreasing pigmentation in skin comprising topically applying to skin in need thereof (e.g., to skin affected by age spots, sun spot, etc.) a composition comprising a topically acceptable vehicle and an effective amount (e.g., from about 0.0001% to about 10% by weight) of an upregulator of any of the genes in Table 1 for a time sufficient (e.g., once or more daily for at least one week) to achieve a reduction in skin pigmentation.

79. The method according to claim 78, wherein said gene is cytochrome P450, family 39, subfamily A, polypeptide 1.

80. The method according to claim 78, wherein said gene is actin, alpha, cardiac muscle 1.

81. The method according to claim 78, wherein said gene is periostin, osteoblast specific factor.

82. The method according to claim 78, wherein said gene is SLIT and NTRK-like family, member 6.

83. The method according to claim 78, wherein said gene is ribosomal protein S6 kinase, 90 kDa, polypeptide 6.

84. The method according to claim 78, wherein said gene is coagulation factor II (thrombin) receptor.

85. The method according to claim 78, wherein said gene is cytochrome P450, family 39, subfamily A, polypeptide 1.

86. The method according to claim 78, wherein said gene is actin, alpha, cardiac muscle 1.

87. The method according to claim 78, wherein said gene is fibronectin leucine rich transmembrane protein 3.

88. The method according to claim 78, wherein said gene is ring finger protein 180.

89. The method according to claim 78, wherein said gene is nicotinamide nucleotide adenylyltransferase 3.

90. The method according to claim 78, wherein said gene is olfactory receptor, family 2, subfamily T, member 2.

91. The method according to claim 78, wherein said gene is zinc finger protein 204, pseudogene.

92. The method according to claim 78, wherein said gene is solute carrier family 38, member 4.

93. The method according to claim 78, wherein said gene is solute carrier family 25, member 27.

94. The method according to claim 78, wherein said gene is kinesin family member 21A.

95. The method according to claim 78, wherein said gene is serine peptidase inhibitor, Kazal type 1.

96. The method according to claim 78, wherein said gene is MX dynamin-like GTPase 2.

97. The method according to claim 78, wherein said gene is chromosome 10 open reading frame 90.

98. The method according to claim 78, wherein said gene is myosin VA (heavy chain 12, myoxin).

99. The method according to claim 78, wherein said gene is plexin C1.

100. The method according to claim 78, wherein said gene is mucolipin 3.

101. The method according to claim 78, wherein said gene is oculocutaneous albinism II.

102. The method according to claim 78, wherein said gene is melan-A.

103. The method according to claim 78, wherein said gene is 2′-5′-oligoadenylate synthetase 2, 69/71 kDa.

104. The method according to claim 78, wherein said gene is G protein-coupled receptor 143.

105. The method according to claim 78, wherein said gene is phosphatidic acid phosphatase type 2 domain containing 1A.

106. The method according to claim 78, wherein said gene is epithelial membrane protein 3.

107. The method according to claim 78, wherein said gene is paired box 3.

108. The method according to claim 78, wherein said gene is solute carrier family 1 (glutamate/neutral amino acid transporter), member 4.

109. The method according to claim 78, wherein said gene is long intergenic non-protein coding RNA 518.

110. The method according to claim 78, wherein said gene is ectonucleotide pyrophosphatase/phosphodiesterase 2.

111. The method according to claim 78, wherein said gene is neuronal regeneration related protein.

112. The method according to claim 78, wherein said gene is CD24 molecule; protocadherin 7.

113. The method according to claim 78, wherein said gene is serpin peptidase inhibitor, clade B (ovalbumin), member 12.

114. The method according to claim 78, wherein said gene is transient receptor potential cation channel, subfamily M, member 1.

115. The method according to claim 78, wherein said gene is endothelin receptor type B.

116. The method according to claim 78, wherein said gene is WD repeat domain 63.

117. The method according to claim 78, wherein said gene is tyrosinase.

118. The method according to claim 78, wherein said gene is tolloid-like 1.

119. The method according to claim 78, wherein said gene is tyrosinase-related protein 1.

120. The method according to claim 78, wherein said gene is premelanosome protein.

121. The method according to claim 78, wherein said gene is hemicentin 1.

122. The method according to claim 78, wherein said gene is homeobox D11.

123. The method according to claim 78, wherein said gene is keratin 6B.

124. The method according to claim 78, wherein said gene is insulin-like growth factor binding protein 3.

125. The method according to claim 78, wherein said gene is Fraser extracellular matrix complex subunit 1.

126. The method according to claim 78, wherein said gene is FRAS1 related extracellular matrix protein 2.

127. The method according to claim 78, wherein said gene is small proline-rich protein 1A.

128. The method according to claim 78, wherein said gene is follicle stimulating hormone receptor.

129. The method according to claim 78, wherein said gene is ATPase, H+/K+ transporting, nongastric, alpha polypeptide.

130. The method according to claim 78, wherein said gene is homeobox D10.

131. A method of increasing pigmentation in skin comprising topically applying to skin in need thereof (e.g., to skin affected by age spots, sun spot, etc.) a composition comprising a topically acceptable vehicle and an effective amount (e.g., from about 0.0001% to about 10% by weight) of a downregulator of any of the genes in Table 1 for a time sufficient (e.g., once or more daily for at least one week) to achieve an increase in skin pigmentation.

132. The method according to claim 131, wherein said gene is cytochrome P450, family 39, subfamily A, polypeptide 1.

133. The method according to claim 131, wherein said gene is actin, alpha, cardiac muscle 1.

134. The method according to claim 131, wherein said gene is periostin, osteoblast specific factor.

135. The method according to claim 131, wherein said gene is SLIT and NTRK-like family, member 6.

136. The method according to claim 131, wherein said gene is ribosomal protein S6 kinase, 90 kDa, polypeptide 6.

137. The method according to claim 131, wherein said gene is coagulation factor II (thrombin) receptor.

138. The method according to claim 131, wherein said gene is cytochrome P450, family 39, subfamily A, polypeptide 1.

139. The method according to claim 131, wherein said gene is actin, alpha, cardiac muscle 1.

140. The method according to claim 131, wherein said gene is fibronectin leucine rich transmembrane protein 3.

141. The method according to claim 131, wherein said gene is ring finger protein 180.

142. The method according to claim 131, wherein said gene is nicotinamide nucleotide adenylyltransferase 3.

143. The method according to claim 131, wherein said gene is olfactory receptor, family 2, subfamily T, member 2.

144. The method according to claim 131, wherein said gene is zinc finger protein 204, pseudogene.

145. The method according to claim 131, wherein said gene is solute carrier family 38, member 4.

146. The method according to claim 131, wherein said gene is solute carrier family 25, member 27.

147. The method according to claim 131, wherein said gene is kinesin family member 21A.

148. The method according to claim 131, wherein said gene is serine peptidase inhibitor, Kazal type 1.

149. The method according to claim 131, wherein said gene is MX dynamin-like GTPase 2.

150. The method according to claim 131, wherein said gene is chromosome 10 open reading frame 90.

151. The method according to claim 131, wherein said gene is myosin VA (heavy chain 12, myoxin).

152. The method according to claim 131, wherein said gene is plexin C1.

153. The method according to claim 131, wherein said gene is mucolipin 3.

154. The method according to claim 131, wherein said gene is oculocutaneous albinism II.

155. The method according to claim 131, wherein said gene is melan-A.

156. The method according to claim 131, wherein said gene is 2′-5′-oligoadenylate synthetase 2, 69/71 kDa.

157. The method according to claim 131, wherein said gene is G protein-coupled receptor 143.

158. The method according to claim 131, wherein said gene is phosphatidic acid phosphatase type 2 domain containing 1A.

159. The method according to claim 131, wherein said gene is epithelial membrane protein 3.

160. The method according to claim 131, wherein said gene is paired box 3.

161. The method according to claim 131, wherein said gene is solute carrier family 1 (glutamate/neutral amino acid transporter), member 4.

162. The method according to claim 131, wherein said gene is long intergenic non-protein coding RNA 518.

163. The method according to claim 131, wherein said gene is ectonucleotide pyrophosphatase/phosphodiesterase 2.

164. The method according to claim 131, wherein said gene is neuronal regeneration related protein.

165. The method according to claim 131, wherein said gene is CD24 molecule; protocadherin 7.

166. The method according to claim 131, wherein said gene is serpin peptidase inhibitor, clade B (ovalbumin), member 12.

167. The method according to claim 131, wherein said gene is transient receptor potential cation channel, subfamily M, member 1.

168. The method according to claim 131, wherein said gene is endothelin receptor type B.

169. The method according to claim 131, wherein said gene is WD repeat domain 63.

170. The method according to claim 131, wherein said gene is tyrosinase.

171. The method according to claim 131, wherein said gene is tolloid-like 1.

172. The method according to claim 131, wherein said gene is tyrosinase-related protein 1.

173. The method according to claim 131, wherein said gene is premelanosome protein.

174. The method according to claim 131, wherein said gene is hemicentin 1.

175. The method according to claim 131, wherein said gene is homeobox D11.

176. The method according to claim 131, wherein said gene is keratin 6B.

177. The method according to claim 131, wherein said gene is insulin-like growth factor binding protein 3.

178. The method according to claim 131, wherein said gene is Fraser extracellular matrix complex subunit 1.

179. The method according to claim 131, wherein said gene is FRAS1 related extracellular matrix protein 2.

180. The method according to claim 131, wherein said gene is small proline-rich protein 1A.

181. The method according to claim 131, wherein said gene is follicle stimulating hormone receptor.

182. The method according to claim 131, wherein said gene is ATPase, H+/K+ transporting, nongastric, alpha polypeptide.

183. The method according to claim 131, wherein said gene is homeobox D10.

184. A method of treating skin comprising topically applying to skin in need thereof (e.g., to skin affected by age spots, sun spot, etc.) a composition comprising a topically acceptable vehicle and an effective amount (e.g., from about 0.0001% to about 10% by weight) of a modulator of any of the genes in Table 1 for a time sufficient (e.g., once or more daily for at least one week) to improve the aesthetic appearance of said skin.

185. The method according to claim 184, wherein said gene is cytochrome P450, family 39, subfamily A, polypeptide 1.

186. The method according to claim 184, wherein said gene is actin, alpha, cardiac muscle 1.

187. The method according to claim 184, wherein said gene is periostin, osteoblast specific factor.

188. The method according to claim 184, wherein said gene is SLIT and NTRK-like family, member 6.

189. The method according to claim 184, wherein said gene is ribosomal protein S6 kinase, 90 kDa, polypeptide 6.

190. The method according to claim 184, wherein said gene is coagulation factor II (thrombin) receptor.

191. The method according to claim 184, wherein said gene is cytochrome P450, family 39, subfamily A, polypeptide 1.

192. The method according to claim 184, wherein said gene is actin, alpha, cardiac muscle 1.

193. The method according to claim 184, wherein said gene is fibronectin leucine rich transmembrane protein 3.

194. The method according to claim 184, wherein said gene is ring finger protein 180.

195. The method according to claim 184, wherein said gene is nicotinamide nucleotide adenylyltransferase 3.

196. The method according to claim 184, wherein said gene is olfactory receptor, family 2, subfamily T, member 2.

197. The method according to claim 184, wherein said gene is zinc finger protein 204, pseudogene.

198. The method according to claim 184, wherein said gene is solute carrier family 38, member 4.

199. The method according to claim 184, wherein said gene is solute carrier family 25, member 27.

200. The method according to claim 184, wherein said gene is kinesin family member 21A.

201. The method according to claim 184, wherein said gene is serine peptidase inhibitor, Kazal type 1.

202. The method according to claim 184, wherein said gene MX dynamin-like GTPase 2.

203. The method according to claim 184, wherein said gene is chromosome 10 open reading frame 90.

204. The method according to claim 184, wherein said gene is myosin VA (heavy chain 12, myoxin).

205. The method according to claim 184, wherein said gene is plexin C1.

206. The method according to claim 184, wherein said gene is mucolipin 3.

207. The method according to claim 184, wherein said gene is oculocutaneous albinism II.

208. The method according to claim 184, wherein said gene is melan-A.

209. The method according to claim 184, wherein said gene is 2′-5′-oligoadenylate synthetase 2, 69/71 kDa.

210. The method according to claim 184, wherein said gene is G protein-coupled receptor 143.

211. The method according to claim 184, wherein said gene is phosphatidic acid phosphatase type 2 domain containing 1A.

212. The method according to claim 184, wherein said gene is epithelial membrane protein 3.

213. The method according to claim 184, wherein said gene is paired box 3.

214. The method according to claim 184, wherein said gene is solute carrier family 1 (glutamate/neutral amino acid transporter), member 4.

215. The method according to claim 184, wherein said gene is long intergenic non-protein coding RNA 518.

216. The method according to claim 184, wherein said gene is ectonucleotide pyrophosphatase/phosphodiesterase 2.

217. The method according to claim 184, wherein said gene is neuronal regeneration related protein.

218. The method according to claim 184, wherein said gene is CD24 molecule; protocadherin 7.

219. The method according to claim 184, wherein said gene is serpin peptidase inhibitor, clade B (ovalbumin), member 12.

220. The method according to claim 184, wherein said gene is transient receptor potential cation channel, subfamily M, member 1.

221. The method according to claim 184, wherein said gene is endothelin receptor type B.

222. The method according to claim 184, wherein said gene is WD repeat domain 63.

223. The method according to claim 184, wherein said gene is tyrosinase.

224. The method according to claim 184, wherein said gene is tolloid-like 1.

225. The method according to claim 184, wherein said gene is tyrosinase-related protein 1.

226. The method according to claim 184, wherein said gene is premelanosome protein.

227. The method according to claim 184, wherein said gene is hemicentin 1.

228. The method according to claim 184, wherein said gene is homeobox D11.

229. The method according to claim 184, wherein said gene is keratin 6B.

230. The method according to claim 184, wherein said gene is insulin-like growth factor binding protein 3.

231. The method according to claim 184, wherein said gene is Fraser extracellular matrix complex subunit 1.

232. The method according to claim 184, wherein said gene is FRAS1 related extracellular matrix protein 2.

233. The method according to claim 184, wherein said gene is small proline-rich protein 1A.

234. The method according to claim 184, wherein said gene is follicle stimulating hormone receptor.

235. The method according to claim 1841, wherein said gene is ATPase, H+/K+ transporting, nongastric, alpha polypeptide.

236. The method according to claim 184, wherein said gene is homeobox D10.

What is claimed is:

1. A method of screening for modulators of skin pigmentation comprising:
(a) contacting a human skin cell with a candidate substance; and
(b) determining whether expression of at least two genes is modulated in said skin cell, said genes being selected from the group consisting of: MX dynamin-like GTPase 2; chromosome 10 open reading frame 90; myosin VA (heavy chain 12, myoxin); plexin C1; mucolipin 3; oculocutaneous albinism II; melan-A; 2′-5′-oligoadenylate synthetase 2, 69/71 kDa; G protein-coupled receptor 143; phosphatidic acid phosphatase type 2 domain containing 1A; epithelial membrane protein 3; paired box 3; solute carrier family 1 (glutamate/neutral amino acid transporter), member 4; long intergenic non-protein coding RNA 518; ectonucleotide pyrophosphatase/phosphodiesterase 2; neuronal regeneration related protein; CD24 molecule; protocadherin 7; serpin peptidase inhibitor, clade B (ovalbumin), member 12; transient receptor potential cation channel, subfamily M, member 1; endothelin receptor type B; WD repeat domain 63; tolloid-like 1; tyrosinase-related protein 1; premelanosome protein; hemicentin 1; homeobox D11; keratin 6B; insulin-like growth factor binding protein 3; Fraser extracellular matrix complex subunit 1; FRAS1 related extracellular matrix protein 2; small proline-rich protein 1A; follicle stimulating hormone receptor; ATPase, H+/K+ transporting, nongastric, alpha polypeptide; homeobox D10; cytochrome P450, family 39, subfamily A, polypeptide 1; actin, alpha, cardiac muscle 1; periostin, osteoblast specific factor; SLIT and NTRK-like family, member 6; ribosomal protein S6 kinase, 90 kDa, polypeptide 6; coagulation factor II (thrombin) receptor; actin, alpha, cardiac muscle 1; fibronectin leucine rich transmembrane protein 3; ring finger protein 180; nicotinamide nucleotide adenylyltransferase 3; olfactory receptor, family 2, subfamily T, member 2; zinc finger protein 204, pseudogene; solute carrier family 38, member 4; solute carrier family 25, member 27; kinesin family member 21A; serine peptidase inhibitor, and Kazal type 1;
wherein modulation of said at least two genes indicates that said candidate substance modulates skin pigmentation.

2. The method according to claim 1, wherein said skin cell is a fibroblast or a keratinocyte.

3. The method according to claim 1, wherein said modulation comprises upregulation or downregulation of said two or more genes.

4. The method according to claim 1, wherein said step of determining further comprises obtaining a skin cell, and extracting nucleic acids from said skin cell.

5. The method according to claim 1, wherein said step of determining further comprises quantifying said nucleic acids extracted from said skin cell.

6. The method according to claim 1, further comprising determining whether expression of said two or more genes is modulated in a skin cell that has not been contacted with said candidate substance.

7. The method according to claim 1, wherein said modulation comprises between about a 1.2-fold change and about a 10-fold change in gene expression relative to expression of the same genes in a skin cell that has not been contacted with said candidate substance.

8. The method according to claim 1, wherein expression of at least three genes is modulated in said skin cell.

9. The method according to claim 1, wherein expression of at least one gene is upregulated and expression of at least one gene is downregulated in said skin cell.

10. A method of reducing skin pigmentation, comprising topically applying to human skin in need thereof a cosmetic composition comprising an effective amount of:
a. a downregulator of one or more of the genes selected from the group consisting of: MX dynamin-like GTPase 2; chromosome 10 open reading frame 90; myosin VA (heavy chain 12, myoxin); plexin C1; mucolipin 3; oculocutaneous albinism II; melan-A; 2′-5′-oligoadenylate synthetase 2, 69/71 kDa; G protein-coupled receptor 143; phosphatidic acid phosphatase type 2 domain containing 1A; epithelial membrane protein 3; paired box 3; solute carrier family 1 (glutamate/neutral amino acid transporter), member 4; long intergenic non-protein coding RNA 518; ectonucleotide pyrophosphatase/phosphodiesterase 2; neuronal regeneration related protein; CD24 molecule; protocadherin 7; serpin peptidase inhibitor, clade B (ovalbumin), member 12; transient receptor potential cation channel, subfamily M, member 1; endothelin receptor type B; WD repeat domain 63; tolloid-like 1; tyrosinase-related protein 1; premelanosome protein; hemicentin 1; homeobox D11; keratin 6B; insulin-like growth factor binding protein 3; Fraser extracellular matrix complex subunit 1; FRAS1 related extracellular matrix protein 2; small proline-rich protein 1A; follicle stimulating hormone receptor; ATPase, H+/K+ transporting, nongastric, alpha polypeptide; and homeobox D10; and/or
b. an upregulator of one or more of the genes selected from the group consisting of: cytochrome P450, family 39, subfamily A, polypeptide 1; actin, alpha, cardiac muscle 1; periostin, osteoblast specific factor; SLIT and NTRK-like family, member 6; ribosomal protein S6 kinase, 90 kDa, polypeptide 6; coagulation factor II (thrombin) receptor; actin, alpha, cardiac muscle 1; fibronectin leucine rich transmembrane protein 3; ring finger protein 180; nicotinamide nucleotide adenylyltransferase 3; olfactory receptor, family 2, subfamily T, member 2; zinc finger protein 204, pseudogene; solute carrier family 38, member 4; solute carrier family 25, member 27; kinesin family member 21A; serine peptidase inhibitor, and Kazal type 1;
wherein said downregulator and/or upregulator of said one or more genes is present in an amount sufficient to decrease pigmentation in said skin.

11. A cosmetic composition for topical application comprising, in a cosmetically acceptable vehicle,
a. a downregulator of one or more of the genes selected from the group consisting of: MX dynamin-like GTPase 2; chromosome 10 open reading frame 90; myosin VA (heavy chain 12, myoxin); plexin C1; mucolipin 3; oculocutaneous albinism II; melan-A; 2′-5′-oligoadenylate synthetase 2, 69/71 kDa; G protein-coupled receptor 143; phosphatidic acid phosphatase type 2 domain containing 1A; epithelial membrane protein 3; paired box 3; solute carrier family 1 (glutamate/neutral amino acid transporter), member 4; long intergenic non-protein coding RNA 518; ectonucleotide pyrophosphatase/phosphodiesterase 2; neuronal regeneration related protein; CD24 molecule; protocadherin 7; serpin peptidase inhibitor, clade B (ovalbumin), member 12; transient receptor potential cation channel, subfamily M, member 1; endothelin receptor type B; WD repeat domain 63; tolloid-like 1; tyrosinase-related protein 1; premelanosome protein; hemicentin 1; homeobox D11; keratin 6B; insulin-like growth factor binding protein 3; Fraser extracellular matrix complex subunit 1; FRAS1 related extracellular matrix protein 2; small proline-rich protein 1A; follicle stimulating hormone receptor; ATPase, H+/K+ transporting, nongastric, alpha polypeptide; and homeobox D10; and/or
b. an upregulator of one or more of the genes selected from the group consisting of: cytochrome P450, family 39, subfamily A, polypeptide 1; actin, alpha, cardiac muscle 1; periostin, osteoblast specific factor; SLIT and NTRK-like family, member 6; ribosomal protein S6 kinase, 90 kDa, polypeptide 6; coagulation factor II (thrombin) receptor; actin, alpha, cardiac muscle 1; fibronectin leucine rich transmembrane protein 3; ring finger protein 180; nicotinamide nucleotide adenylyltransferase 3; olfactory receptor, family 2, subfamily T, member 2; zinc finger protein 204, pseudogene; solute carrier family 38, member 4; solute carrier family 25, member 27; kinesin family member 21A; serine peptidase inhibitor, and Kazal type 1.

12. A gene panel, wherein at least 50% of nucleic acids on said gene panel comprise oligonucleotides that hybridize with nucleic acids corresponding to genes selected from the group consisting of: cytochrome P450, family 39, subfamily A, polypeptide 1; actin, alpha, cardiac muscle 1; periostin, osteoblast specific factor; SLIT and NTRK-like family, member 6; ribosomal protein S6 kinase, 90 kDa, polypeptide 6; coagulation factor II (thrombin) receptor; actin, alpha, cardiac muscle 1; fibronectin leucine rich transmembrane protein 3; ring finger protein 180; nicotinamide nucleotide adenylyltransferase 3; olfactory receptor, family 2, subfamily T, member 2; zinc finger protein 204, pseudogene; solute carrier family 38, member 4; solute carrier family 25, member 27; kinesin family member 21A; serine peptidase inhibitor, Kazal type 1; MX dynamin-like GTPase 2; chromosome 10 open reading frame 90; myosin VA (heavy chain 12, myoxin); plexin C1; mucolipin 3; oculocutaneous albinism II; melan-A; 2′-5′-oligoadenylate synthetase 2, 69/71 kDa; G protein-coupled receptor 143; phosphatidic acid phosphatase type 2 domain containing 1A; epithelial membrane protein 3; paired box 3; solute carrier family 1 (glutamate/neutral amino acid transporter), member 4; long intergenic non-protein coding RNA 518; ectonucleotide pyrophosphatase/phosphodiesterase 2; neuronal regeneration related protein; CD24 molecule; protocadherin 7; serpin peptidase inhibitor, clade B (ovalbumin), member 12; transient receptor potential cation channel, subfamily M, member 1; endothelin receptor type B; WD repeat domain 63; tyrosinase; tolloid-like 1; tyrosinase-related protein 1; premelanosome protein; hemicentin 1; homeobox D11; keratin 6B; insulin-like growth factor binding protein 3; Fraser extracellular matrix complex subunit 1; FRAS1 related extracellular matrix protein 2; small proline-rich protein 1A; follicle stimulating hormone receptor; ATPase, H+/K+ transporting, nongastric, alpha polypeptide; and homeobox D10.

13. A gene panel, wherein at least 75% of nucleic acids on said gene panel comprise oligonucleotides that hybridize with nucleic acids corresponding to genes selected from the group consisting of: cytochrome P450, family 39, subfamily A, polypeptide 1; actin, alpha, cardiac muscle 1; periostin, osteoblast specific factor; SLIT and NTRK-like family, member 6; ribosomal protein S6 kinase, 90 kDa, polypeptide 6; coagulation factor II (thrombin) receptor; actin, alpha, cardiac muscle 1; fibronectin leucine rich transmembrane protein 3; ring finger protein 180; nicotinamide nucleotide adenylyltransferase 3; olfactory receptor, family 2, subfamily T, member 2; zinc finger protein 204, pseudogene; solute carrier family 38, member 4; solute carrier family 25, member 27; kinesin family member 21A; serine peptidase inhibitor, Kazal type 1; MX dynamin-like GTPase 2; chromosome 10 open reading frame 90; myosin VA (heavy chain 12, myoxin); plexin C1; mucolipin 3; oculocutaneous albinism II; melan-A; 2′-5′-oligoadenylate synthetase 2, 69/71 kDa; G protein-coupled receptor 143; phosphatidic acid phosphatase type 2 domain containing 1A; epithelial membrane protein 3; paired box 3; solute carrier family 1 (glutamate/neutral amino acid transporter), member 4; long intergenic non-protein coding RNA 518; ectonucleotide pyrophosphatase/phosphodiesterase 2; neuronal regeneration related protein; CD24 molecule; protocadherin 7; serpin peptidase inhibitor, clade B (ovalbumin), member 12; transient receptor potential cation channel, subfamily M, member 1; endothelin receptor type B; WD repeat domain 63; tyrosinase; tolloid-like 1; tyrosinase-related protein 1; premelanosome protein; hemicentin 1; homeobox D11; keratin 6B; insulin-like growth factor binding protein 3; Fraser extracellular matrix complex subunit 1; FRAS1 related extracellular matrix protein 2; small proline-rich protein 1A; follicle stimulating hormone receptor; ATPase, H+/K+ transporting, nongastric, alpha polypeptide; and homeobox D10.

14. The gene panel of claim 13, wherein at least 90% of nucleic acids on said gene panel comprise oligonucleotides that hybridize with nucleic acids corresponding to genes selected from the group.
